(19) United States
(12) Patent Application Publication
(10) Pub. No.: US 2006/0135418 A1
(43) Pub. Date: Jun. 22, 2006

(54) Receptors

(75) Inventors: Bent Karsten Jakobsen, Abingdon (GB); Meir Glick, Stoughton, MA (US)

Correspondence Address: BANNER & WITCOFF, 1001 G STREET N W, SUITE 1100, WASHINGTON, DC, 20001, US

(73) Assignee: Avidex Ltd., Abingdon (GB)

(21) Appl. No.: 10/535,965

(22) PCT Filed: Nov. 25, 2003
(86) PCT No.: PCT/GB03/05104

§ 371 Date: Oct. 20, 2005

Related U.S. Application Data
(60) Provisional application No. 60/475,906, filed on Jun. 5, 2003.

(30) Foreign Application Priority Data
Dec. 3, 2002 (GB) 0228112.9
Feb. 22, 2003 (GB) 0304090.4
Apr. 10, 2003 (GB) 0308309.4
Jun. 18, 2003 (GB) 0314113.2
Jul. 11, 2003 (GB) 0316354.0

(51) Int. Cl. A61K 38/17; C07K 14/705

(52) U.S. Cl. 514/12; 530/350

(57) ABSTRACT

A multivalent T cell receptor (TCR) complex comprising at least two TCRs, linked by a non-peptidic polymer chain or a peptidic linker sequence. Preferably the TCR complex comprises TCR heterodimers having a non-native disulfide bond between constant domain residues, said TCRs being linked via an optionally substituted, polyalkylene glycol linker. Therapeutic agents such as cytotoxic drugs may be attached to such complexes for targeted cell delivery. Such TCR complexes may be used in the diagnosis or treatment of cancer, infectious disease, or autoimmune disease.

The present invention relates to a multivalent T cell receptor complex comprising at least two T cell receptors linked by a non-peptidic polymer chain or a peptidic linker sequence, and to the use of such complexes in medicine, particularly the diagnosis and treatment of autoimmune disease and cancer.

### BACKGROUND TO THE INVENTION

Native TCRs

As is described in, for example, WO 99/60120 TCRs mediate the recognition of specific Major Histocompatibility Complex (MHC)-peptide complexes by T cells and, as such, are essential to the functioning of the cellular arm of the immune system.

Antibodies and TCRs are the only two types of molecules which recognise antigens in a specific manner, and thus the TCR is the only receptor for particular peptide antigens presented in MHC, the alien peptide often being the only sign of an abnormality within a cell. T cell recognition occurs when a T-cell and an antigen presenting cell (APC) are in direct physical contact, and is initiated by ligation of antigen-specific TCRs with peptide-MHC (pMHC) complexes.

The native TCR is a heterodimeric cell surface protein of the immunoglobulin superfamily which is associated with invariant proteins of the CD3 complex involved in mediating signal transduction. TCRs exist in αβ and γδ forms, which are structurally similar but have quite distinct anatomical locations and probably functions. The MHC class I and class II ligands are also immunoglobulin superfamily proteins but are specialised for antigen presentation, with a highly polymorphic peptide binding site which enables them to present a diverse array of short peptide fragments at the APC cell surface.

Two further classes of proteins are known to be capable of functioning as TCR ligands. (1) CD1 antigens are MHC class I-related molecules whose genes are located on a different chromosome from the classical MHC class I and class II antigens. CD1 molecules are capable of presenting peptide and non-peptide (eg lipid, glycolipid) moieties to T cells in a manner analogous to conventional class I and class II-MHC-pep complexes. See, for example (Barclay et al, (1997) The Leucocyte Antigen Factsbook 2^{nd} Edition, Acadmeic Press) and (Bauer (1997) Eur J Immunol 27 (6) 1366-1373)) (2) Bacterial superantigens are soluble toxins which are capable of binding both class II MHC molecules and a subset of TCRs. (Fraser (1989) Nature 339 221-223) Many superantigens exhibit specificity for one or two Vbeta segments, whereas others exhibit more promiscuous binding. In any event, superantigens are capable of eliciting an enhanced immune response by virtue of their ability to stimulate subsets of T cells in a polyclonal fashion.

The extracellular portion of native heterodimeric αβ and γδ TCRs consists of two polypeptides each of which has a membrane-proximal constant domain, and a membrane-distal variable domain. Each of the constant and variable domains includes an intra-chain disulfide bond. The variable domains contain the highly polymorphic loops analogous to the complementarity determining regions (CDRs) of antibodies. CDR3 of αβ TCRs interact with the peptide presented by MHC, and CDRs 1 and 2 of αβ TCRs interact with the peptide and the MHC. The diversity of TCR sequences is generated via somatic rearrangement of linked variable (V), diversity (D), joining (J), and constant (C) genes

Functional α and γ chain polypeptides are formed by rearranged V-J-C regions, whereas β and δ chains consist of V-D-J-C regions. The extracellular constant domain has a membrane proximal region and an immunoglobulin region. There are single α and δ chain constant domains, known as TRAC and TRDC respectively. The β chain constant domain is composed of one of two different β constant domains, known as TRBC1 and TRBC2 (IMGT nomenclature). There are four amino acid changes between these β constant domains, three of which are within the domains used to produce the single-chain TCRs displayed on phage particles of the present invention. These changes are all within exon 1 of TRBC1 and TRBC2: N₄K₅->K₄N₅ and F₃₇->Y (IMGT numbering, differences TRBC1->TRBC2), the final amino acid change between the two TCR β chain constant regions being in exon 3 of TRBC1 and TRBC2: V₁->E. The constant γ domain is composed of one of either TRGC1, TRGC2(2×) or TRGC2(3×). The two TRGC2 constant domains differ only in the number of copies of the amino acids encoded by exon 2 of this gene that are present.

The extent of each of the TCR extracellular domains is somewhat variable. However, a person skilled in the art can readily determine the position of the domain boundaries using a reference such as The T Cell Receptor Facts Book, Lefranc & Lefranc, Publ. Academic Press 2001.

Recombinant TCRs

The production of recombinant TCRs is beneficial as these provide soluble TCR analogues suitable for the following purposes:

Studying the TCR/ligand interactions (e.g. pMHC for αβ TCRs)

Screening for inhibitors of TCR-associated interactions

Providing the basis for potential therapeutics

A number of constructs have been devised to date for the production of recombinant TCRs. These constructs fall into two broad classes, single-chain TCRs and dimeric TCRs, and the literature relevant to these constructs is summarised below.

Single Chain TCRs

Single-chain TCRs (scTCRs) are artificial constructs consisting of a single amino acid strand, which like native heterodimeric TCRs bind to MHC-peptide complexes. Unfortunately, attempts to produce functional alpha/beta analogue scTCRs by simply linking the alpha and beta chains such that both are expressed in a single open reading frame have been unsuccessful, presumably because of the natural instability of the alpha-beta soluble domain pairing.

Accordingly, special techniques using various truncations of either or both of the alpha and beta chains have been necessary for the production of scTCRs. These formats appear to be applicable only to a very limited range of scTCR sequences. Soo Hoo et al (1992) PNAS. 89 (10): 4759-63 report the expression of a mouse TCR in single chain format from the 2C T cell clone using a truncated beta and alpha chain linked with a 25 amino acid linker and bacterial periplasmic expression (see also Schodin et al (1996) Mol. Immunol. 33 (9): 819-29). This design also forms the basis of the m6 single-chain TCR reported by Holler et al (2000) PNAS. 97 (10): 5387-92 which is derived from the 2C scTCR and binds to the same H2-Ld-restricted alloepitope. Shusta et al (2000) Nature Biotechnology 18: 754-759 report using single-chain 2 C TCR constructs in yeast display experiments, which produced mutated TCRs with, enhanced thermal stability and solubility. This report also demonstrated the ability of these displayed 2C TCRs to selectively bind cells expressing their cognate pMHC. Khandekar et al (1997) J. Biol. Chem. 272 (51): 32190-7 report a similar design for the murine D10 TCR, although this scTCR was fused to MBP and expressed in bacterial cytoplasm (see also Hare et al (1999) Nat. Struct. Biol. 6 (6): 574-81). Hilyard et al (1994) PNAS. 91 (19): 9057-61 report a human scTCR specific for influenza matrix protein-HLA-A2, using a Vα-linker-Vβ design and expressed in bacterial periplasm.

Chung et al (1994) PNAS. 91 (26) 12654-8 report the production of a human scTCR using a Vα-linker-Vβ-Cβ design and expression on the surface of a mammalian cell line. This report does not include any reference to peptide-HLA specific binding of the scTCR. Plaksin et al (1997) J. Immunol. 158 (5): 2218-27 report a similar Vα-linker-Vβ-Cβ design for producing a murine scTCR specific for an HIV gp120-H-2D^{d} epitope. This scTCR is expressed as bacterial inclusion bodies and refolded in vitro.

Dimeric TCRs

A number of papers describe the production of TCR heterodimers which include the native disulphide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840). However, although such TCRs can be recognised by TCR-specific antibodies, none were shown to recognise its native ligand at anything other than relatively high concentrations and/or were not stable.

In WO 99/60120, a soluble TCR is described which is correctly folded so that it is capable of recognising its native ligand, is stable over a period of time, and can be produced in reasonable quantities. This TCR comprises a TCR α or γ chain extracellular domain dimerised to a TCR β or δ chain extracellular domain respectively, by means of a pair of C-terminal dimerisation peptides, such as leucine zippers. This strategy of producing TCRs is generally applicable to all TCRs.

Guillaume et al, Nature Immunology, 2003 (advance on-line publication), details the construction of a soluble JM22 TCR containing an introduced disulphide inter-chain bond between amino acids attached to the C terminus of the construct. This particular construct was derived from the extracellular portion of the JM22 TCR, truncated a single amino acid N terminal to the position of the native disulphide inter-chain bond. C terminal constant domain extensions were added to both the α and β chains of this TCR. These extensions caused the position of the inter-chain forming cysteine residues to be displaced downstream by three amino acids in the α chain and six amino acids in the β chain relative to their native positions. Soluble TCRs of this general design, that is soluble TCRs comprising introduced C terminal constant domain extensions containing a disulphide inter-chain disulphide bond, may also be used in multivalent TCR complexes of the present invention.

Reiter et al, Immunity, 1995, 2:281-287, details the construction of a soluble molecule comprising disulphide-stabilised TCR α and β variable domains, one of which is linked to a truncated form of Pseudomonas exotoxin (PE38). One of the stated reasons for producing this molecule was to overcome the inherent instability of single-chain TCRs. The position of the novel disulphide bond in the TCR variable domains was identified via homology with the variable domains of antibodies, into which these have previously been introduced (for example see Brinkmann, et al. (1993), Proc. Natl. Acad. Sci. USA 90: 7538-7542, and Reiter, et al. (1994) Biochemistry 33: 5451-5459). However, as there is no such homology between antibody and TCR constant domains, such a technique could not be employed to identify appropriate sites for new inter-chain disulphide bonds between TCR constant domains.

Therapeutic and Diagnostic Use

There is a need for TCRs as targeting moieties capable of localising to cells affected by disease processes. Such targeting moieties could be utilised either to directly block the ‘miss-directed’ action of the immune system responsible for auto-immune disease or as a means of delivering cytotoxic agents to cancerous cells. Such molecules should have good affinities for their target ligands and adequate plasma stabilities.

### BRIEF DESCRIPTION OF THE INVENTION

This invention makes available novel multivalent TCR complexes having an increased plasma half-life and improved affinity for their cognate ligands compared to the corresponding monovalent TCR molecules. In the complexes of the invention, the TCRs are linked by non-peptidic polymer chains or by peptidic linkers. The TCRs in the complexes may be scTCRs or dTCRs.

### DETAILED DESCRIPTION OF THE INVENTION

The present invention provides a multivalent T cell receptor (TCR) complex comprising at least two TCRs, linked by a non-peptidic polymer chain or a peptidic linker sequence.

Preferably, the TCRs in the complex are constituted by amino acid sequences corresponding to extracellular constant and variable region sequences of native TCRs

Preferably the polymer chain or peptidic linker sequence extends between amino acid residues of each TCR which are not located in a variable region sequence of the TCR.

The TCRs in the complex may be linked by, for example a polyalkylene glycol chain such as a polyethylene glycol chain, or a peptidic linker derived from a human multimerisation domain.

In one embodiment of the invention a divalent alkylene spacer radical, for example a —CH₂— or —CH₂CH₂—.radical, is located between the polyalkylene glycol chain and its point of attachment to a TCR of the complex.

Multimeric TCR complexes of the invention may be, for example divalent, trivalent or tetravalent, but divalent complexes (ie those which contain only two TCRs) are presently preferred.

Preferred TCRs Present in the Complexes of the Invention

In the complexes of the invention, the TCR molecules may be single chain T cell receptor (scTCR) polypeptides or, preferably, dimeric TCR (dTCR) polypeptide pairs. scTCR polypeptide, or dTCR polypeptide pairs may be constituted by TCR amino acid sequences corresponding to TCR extracellular constant and variable region sequences, with a variable region sequence of the scTCR corresponding to a variable region sequence of one TCR chain being linked by a linker sequence to a constant region sequence corresponding to a constant region sequence of another TCR chain; the variable region sequences of the dTCR polypeptide pair or scTCR polypeptide are mutually orientated substantially as in native TCRs; and in the case of the scTCR polypeptide a disulfide bond which has no equivalent in native T cell receptors links residues of the polypeptide.

For αβ-analogue scTCRs or dTCRs present in the complexes of the invention, the requirement that the variable region sequences of the α and β segments are mutually orientated substantially as in native αβ T cell receptors is tested by confirming that the molecule binds to the relevant TCR ligand (pMHC complex, CD1-antigen complex, superantigen or superantigen/pMHC complex)—if it binds, then the requirement is met. Interactions with pMHC complexes can be measured using a BIAcore 3000™ or BIAcore 2000™ instrument. WO99/6120 provides detailed descriptions of the methods required to analyse TCR binding to MHC-peptide complexes. These methods are equally applicable to the study of TCR/CD1 and TCR/superantigen interactions. In order to apply these methods to the study of TCR/CD1 interactions soluble forms of CD1 are required, the production of which are described in (Bauer (1997) Eur J Immunol 27 (6) 1366-1373). In the case of γδ-analogue TCRs present in the complexes of the invention the cognate ligands for these molecules are unknown therefore secondary means of verifying the conformation of these molecules such as recognition by antibodies can be employed. The monoclonal antibody MCA991T (available from Serotec), specific for the δ chain variable region, is an example of an antibody appropriate for this task.

scTCR polypeptides present in the complexes of the invention are preferably those which have, for example, a first segment constituted by an amino acid sequence corresponding to a TCR α or δ chain variable region sequence fused to the N terminus of an amino acid sequence corresponding to a TCR α chain constant region extracellular sequence, a second segment constituted by an amino acid sequence corresponding to a TCR β or γ chain variable region fused to the N terminus of an amino acid sequence corresponding to TCR β chain constant region extracellular sequence, a linker sequence linking the C terminus of the first segment to the N terminus of the second segment, or vice versa, and a disulfide bond between the first and second chains, said disulfide bond being one which has no equivalent in native αβ or γδ T cell receptors, the length of the linker sequence and the position of the disulfide bond being such that the variable region sequences of the first and second segments are mutually orientated substantially as in native αβ or γδ T cell receptors.

dTCRs present in the complexes of the invention are preferably those which are constituted by a first polypeptide wherein a sequence corresponding to a TCR α or δ chain variable region sequence is fused to the N terminus of a sequence corresponding to a TCR α chain constant region extracellular sequence, and a second polypeptide wherein a sequence corresponding to a TCR β or γ chain variable region sequence fused to the N terminus a sequence corresponding to a TCR β chain constant region extracellular sequence, the first and second polypeptides being linked by a disulfide bond which has no equivalent in native αβ or γδ T cell receptors.

The constant region extracellular sequences present in the above preferred scTCRs or dTCRs preferably correspond to those of a human TCR, as do the variable region sequences. However, the correspondence between such sequences need not be 1:1 on an amino acid level. N- or C-truncation, and/or amino acid deletion and/or substitution relative to the corresponding human TCR sequences is acceptable, provided the overall result is mutual orientation of the α and β variable region sequences, or γ and δ variable region sequences is as in native αβ, or γδ T cell receptors respectively. In particular, because the constant region extracellular sequences present in the first and second segments are not directly involved in contacts with the ligand to which the scTCR or dTCR binds, they may be shorter than, or may contain substitutions or deletions relative to, extracellular constant domain sequences of native TCRs.

The constant region extracellular sequence present in one of the dTCR polypeptide pair, or in the first segment of a scTCR polypeptide may include a sequence corresponding to the extracellular constant Ig domain of a native TCR α chain, and/or the constant region extracellular sequence present in the other member of the pair or second segment may include a sequence corresponding to the extracellular constant Ig domain of a native TCR β chain.

One member of the polypeptide pair or the first segment of the scTCR polypeptide may correspond to substantially all the variable region of a TCR α chain fused to the N terminus of substantially all the extracellular domain of the constant region of an TCR α chain; and/or the other member of the pair or second segment corresponds to substantially all the variable region of a TCR β chain fused to the N terminus of substantially all the extracellular domain of the constant region of a TCR β chain.

The constant region extracellular sequences present in the dTCR polypeptide pair or first and second segments of the scTCR polypeptide may correspond to the constant regions of the α and β chains of a native TCR truncated at their C termini such that the cysteine residues which form the native inter-chain disulfide bond of the TCR are excluded. Alternatively those cysteine residues may be substituted by another amino acid residue such as serine or alanine, so that the native disulfide bond is deleted. In addition, the native TCR β chain contains an unpaired cysteine residue and that residue may be deleted from, or replaced by a non-cysteine residue in, the β sequence of the scTCR of the invention.

The TCR α and β chain variable region sequences present in the dTCR polypeptide pair or first and second segments of the scTCR polypeptide may together correspond to the functional variable domain of a first TCR, and the TCR α and β chain constant region extracellular sequences present in the dTCR polypeptide pair or first and second segments of the scTCR polypeptide may correspond to those of a second TCR, the first and second TCRs being from the same species. Thus the α and β chain variable region sequences present in dTCR polypeptide pair or first and second segments of the scTCR polypeptide may correspond to those of a first human TCR, and the α and β chain constant region extracellular sequences may correspond to those of a second human TCR. For example, A6 Tax sTCR constant region extracellular sequences can be used as a framework onto which heterologous α and β variable domains can be fused.

The TCR δ and γ chain variable region sequences present in the dTCR polypeptide pair or first and second segments of the scTCR polypeptide respectively, may together correspond to the functional variable domain of a first TCR, and the TCR α and β chain constant region extracellular sequences present in the dTCR polypeptide pair or first and second segments of the scTCR polypeptide respectively, may correspond to those of a second TCR, the first and second TCRs being from the same species. Thus the δ and γ chain variable region sequences present in the dTCR polypeptide pair or first and second segments of the scTCR polypeptide may correspond to those of a first human TCR, and the α and β chain constant region extracellular sequences may correspond to those of a second human TCR. For example, A6 Tax sTCR constant region extracellular sequences can be used as a framework onto which heterologous γ and δ variable domains can be fused.

The TCR α and β, or δ and γ chain variable region sequences present in the dTCR polypeptide pair or first and second segments of the scTCR polypeptide may together correspond to the functional variable domain of a first human TCR, and the TCR α and β chain constant region extracellular sequences present in the dTCR polypeptide pair or first and second segments of the scTCR polypeptide may correspond to those of a second non-human TCR, Thus the α and β, or δ and γ chain variable region sequences present dTCR polypeptide pair or first and second segments of the scTCR polypeptide may correspond to those of a first human TCR, and the α and β chain constant region extracellular sequences may correspond to those of a second non-human TCR. For example, murine TCR constant region extracellular sequences can be used as a framework onto which heterologous human α and β TCR variable domains can be fused.

In a scTCR, a linker sequence may link the first and second TCR segments, to form a single polypeptide strand. The linker sequence may, for example, have the formula -P-AA-P- wherein P is proline and AA represents an amino acid sequence wherein the amino acids are glycine and serine.

For an scTCR to bind to a cognate ligand, eg a MHC-peptide complex or CD1-antigen complex in the case of an αβ TCR, the first and second segments must be paired so that the variable region sequences thereof are orientated for such binding. Hence the linker should have sufficient length to span the distance between the C terminus of the first segment and the N terminus of the second segment, or vice versa. On the other hand excessive linker length should preferably be avoided, in case the end of the linker at the N-terminal variable region sequence blocks or reduces bonding of the scTCR to the target ligand.

For example, in the case where the constant region extracellular sequences present in the first and second segments correspond to the constant regions of the α and β chains of a native TCR truncated at their C termini such that the cysteine residues which form the native interchain disulfide bond of the TCR are excluded, and the linker sequence links the C terminus of the first segment to the N terminus of the second segment, the linker may consist of from 26 to 41, for example 29, 30, 31 or 32 or 33, 34, 35 or 36 amino acids, and a particular linker has the formula -PGGG-(SGGGG)₅-P- or -PGGG-(SGGGG)₆-P- wherein P is proline, G is glycine and S is serine.

A principle characterising feature of the scTCRs of the present complexes, and preferably a feature of the dTCRs, is a disulfide bond between the constant region extracellular sequences of the dTCR polypeptide pair or first and second segments of the scTCR polypeptide. That bond may correspond to the native inter-chain disulfide bond present in native dimeric αβ TCRs, or may have no counterpart in native TCRs, being between cysteines specifically incorporated into the constant region extracellular sequences of dTCR polypeptide pair or first and second segments of the scTCR polypeptide. In some cases, both a native and a non-native disulfide bond may be desirable.

The position of the disulfide bond is subject to the requirement that the variable region sequences of dTCR polypeptide pair or first and second segments of the scTCR polypeptide are mutually orientated substantially as in native αβ or γδ T cell receptors.

The disulfide bond may be formed by mutating non-cysteine residues on the first and second segments to cysteine, and causing the bond to be formed between the mutated residues. Residues whose respective β carbons are approximately 6 Å (0.6 nm) or less, and preferably in the range 3.5 Å (0.35 nm) to 5.9 Å (0.59 nm) apart in the native TCR are preferred, such that a disulfide bond can be formed between cysteine residues introduced in place of the native residues. It is preferred if the disulfide bond is between residues in the constant immunoglobulin region, although it could be between residues of the membrane proximal region. Preferred sites where cysteines can be introduced to form the disulfide bond are the following residues in exon 1 of TRAC*01 for the TCR α chain and TRBC1*01 or TRBC2*01 for the TCR β chain:

| | | Native β carbon |
|---|---|---|
| TCR α chain | TCR β chain | separation (nm) |
| Thr 48 | Ser 57 | 0.473 |
| Thr 45 | Ser 77 | 0.533 |
| Tyr 10 | Ser 17 | 0.359 |
| Thr 45 | Asp 59 | 0.560 |
| Ser 15 | Glu 15 | 0.59 |

Now that the residues in human TCRs which can be mutated into cysteine residues to form a new interchain disulfide bond in dTCRs or scTCRs displayed according to the invention have been identified, those of skill in the art will be able to mutate TCRs of other species in the same way to produce a dTCR or scTCR of that species for phage display. In humans, the skilled person merely needs to look for the following motifs in the respective TCR chains to identify the residue to be mutated (the shaded residue is the residue for mutation to a cysteine).

[image]

In other species, the TCR chains may not have a region which has 100% identity to the above motifs. However, those of skill in the art will be able to use the above motifs to identify the equivalent part of the TCR α or β chain and hence the residue to be mutated to cysteine. Alignment techniques may be used in this respect. For example, ClustalW, available on the European Bioinformatics Institute website (http://www.ebi.ac.uk/index.html) can be used to compare the motifs above to a particular TCR chain sequence in order to locate the relevant part of the TCR sequence for mutation.

The present invention includes within its scope complexes of αβ and γδ-analogue scTCRs, as well as those of other mammals, including, but not limited to, mouse, rat, pig, goat and sheep. As mentioned above, those of skill in the art will be able to determine sites equivalent to the above-described human sites at which cysteine residues can be introduced to form an inter-chain disulfide bond. For example, the following shows the amino acid sequences of the mouse Cα and Cβ soluble domains, together with motifs showing the murine residues equivalent to the human residues mentioned above that can be mutated to cysteines to form a TCR interchain disulfide bond (where the relevant residues are shaded):

Mouse Cα soluble domain:

PYIQNPEPAVYQLKDPRSQDSTLCLFTDFDSQINVPKTMESGTFITDKTV
LDMKAMDSKSNGAIAWSNQTSFTCQDIFKETNATYPSSDVP

Mouse Cβ soluble domain:

ELDRNVTPPKVSLFEPSKAEIANKQKATLVCLARGFFPDHVELSWWVNGR
EVHSGVSTDPQAYKESNYSYCLSSRLRVSATFWHNPRNFRCQVQFHGLSE
EDKWPEGSPKPVTQNISAEAWGRAD

[image]

[image]

As discussed above, the A6 Tax sTCR extracellular constant regions can be used as framework onto which heterologous variable domains can be fused. It is preferred that the heterologous variable region sequences are linked to the constant region sequences at any point between the disulfide bond and the N termini of the constant region sequences. In the case of the A6 Tax TCR α and β constant region sequences, the disulfide bond may be formed between cysteine residues introduced at amino acid residues 158 and 172 respectively. Therefore it is preferred if the heterologous α and β chain variable region sequence attachment points are between residues 159 or 173 and the N terminus of the α or β constant region sequences respectively.

A label or another moiety, such as a toxic or therapeutic moiety, may be included in a multivalent TCR complex of the present invention. For example, the label or other moiety may be included in a mixed molecule multimer. An example of such a multimeric molecule is a tetramer containing three TCR molecules and one peroxidase molecule. This could be achieved by mixing the TCR and the enzyme at a molar ratio of 3:1 to generate tetrameric complexes, and isolating the desired complex from any complexes not containing the correct ratio of molecules. These mixed molecules could contain any combination of molecules, provided that steric hindrance does not compromise or does not significantly compromise the desired function of the molecules. The positioning of the binding sites on the streptavidin molecule is suitable for mixed tetramers since steric hindrance is not likely to occur.

The TCR complex of the present invention may bind a peptide MHC complex, or a given MHC type or types, or a superantigen or a peptide- MHC/superantigen complex, or a CD1-antigen complex.

One embodiment the present invention makes available TCR complexes comprising high affinity TCRs.

As used herein the term ‘high affinity TCR’ refers to a mutated scTCR or dTCR which interacts with a specific TCR ligand and either: has a Kd for the said TCR ligand less than that of a corresponding native TCR as measured by Surface Plasmon Resonance, or has an off-rate (k_{off}) for the said TCR ligand less than that of a corresponding native TCR as measured by Surface Plasmon Resonance.

High affinity scTCRs or dTCRs present in complexes of the present invention are preferably mutated relative to the native TCR in at least one complementarity determining region and/or framework region.

In a preferred embodiment of the invention the TCR complex comprises at least two dTCR polypeptide pairs linked by a polyalkylene glycol chain, wherein a divalent alkylene spacer radical is optionally located between the polyalkylene glycol chain and its point of attachment to a dTCR of the complex, and wherein each said dTCR pair is constituted by:
a first polypeptide wherein a sequence corresponding to a TCR α chain variable domain sequence is fused to the N terminus of a sequence corresponding to a TCR α chain constant domain extracellular sequence, and
a second polypeptide wherein a sequence corresponding to a TCR β chain variable domain sequence is fused to the N terminus a sequence corresponding to a TCR β chain constant domain extracellular sequence,
   the first and second polypeptides being linked by a disulfide bond between cysteine residues substituted for Thr 48 of exon 1 of TRAC*01 and Ser 57 of exon 1 of TRBC1*01 or TRBC2*01 or the non-human equivalent thereof, the point of attachment of the polyalkylene linker to each dTCR of the complex being via the thiol group of a cysteine residue at the C-terminus of the dTCR.
   TCR Linkers

In the complexes of the invention, at least two TCR molecules are linked via linker moieties to form multivalent complexes. The TCRs are linked by non-peptidic polymer chains or by peptidic linker sequences. Preferably the complexes are water soluble, so the linker moiety should be selected accordingly. Furthermore, it is preferable that the linker moiety should be capable of attachment to defined positions on the TCR molecules, so that the structural diversity of the complexes formed is minimised. Since the complexes of the invention may be for use in medicine, the linker moieties should be chosen with due regard to their pharmaceutical suitability, for example their immunogenicity.

Examples of linker moieties which fulfil the above desirable criteria are known in the art, for example the art of linking antibody fragments.

There are two classes of linker that are preferred for use in the production of multivalent TCR molecules of the present invention. The first are hydrophilic polymers such as polyalkylene glycols. The most commonly used of this class are based on polyethylene glycol or PEG, the structure of which is shown below.
HOCH₂CH₂O(CH₂CH₂O)ₙ—CH₂CH₂OH
wherein n is about 3 to about 3500. However, others are based on other suitable, optionally substituted, polyalkylene glycols include polypropylene glycol, and copolymers of ethylene glycol and propylene glycol.

Such polymers may be used to treat or conjugate therapeutic agents, particularly polypeptide or protein therapeutics, to achieve beneficial changes to the PK profile of the therapeutic, for example reduced renal clearance, improved plasma half-life, reduced immunogenicity, and improved solubility. Such improvements in the PK profile of the PEG-therapeutic conjugate are believe to result from the PEG molecule or molecules forming a ‘shell’ around the therapeutic which sterically hinders the reaction with the immune system and reduces proteolytic degradation. (Casey et al, (2000) Tumor Targetting 4 235-244) The size of the hydrophilic polymer used my in particular be selected on the basis of the intended therapeutic use of the TCR complex. Thus for example, where the product is intended to leave the circulation and penetrate tissue, for example for use in the treatment of a tumour, it may be advantageous to use low molecular weight polymers in the order of 5 KDa. There are numerous review papers and books that detail the use of PEG and similar molecules in pharmaceutical formulations. For example, see Harris (1992) Polyethylene Glycol Chemistry—Biotechnical and Biomedical Applications, Plenum, New York, N.Y. or Harris & Zalipsky (1997) Chemistry and Biological Applications of Polyethylene Glycol ACS Books, Washington, D.C.

The polymer used can have a linear or branched conformation. Branched PEG molecules, or derivatives thereof, can be induced by the addition of branching moieties including glycerol and glycerol oligomers, pentaerythritol, sorbitol and lysine.

Usually, the polymer will have a chemically reactive group or groups in its structure, for example at one or both termini, and/or on branches from the backbone, to enable the polymer to link to target sites in the TCR. This chemically reactive group or groups may be attached directly to the hydrophilic polymer, or there may be a spacer group/moiety between the hydrophilic polymer and the reactive chemistry as shown below:

Reactive chemistry-Hydrophilic polymer-Reactive chemistry

Reactive chemistry-Spacer-Hydrophilic polymer-Spacer-Reactive chemistry

The spacer used in the formation of constructs of the type outlined above may be any organic moiety that is a non-reactive, chemically stable, chain, Such spacers include, by are not limited to the following:
—(CH₂)ₙ— wherein n=2 to 5
—(CH₂)₃NHCO(CH₂)₂

There are a number of commercial suppliers of hydrophilic polymers linked, directly or via a spacer, to reactive chemistries that may be of use in the present invention. These suppliers include Nektar Therapeutics (CA, USA), NOF Corporation (Japan), Sunbio (South Korea) and Enzon Pharmaceuticals (NJ, USA).

Commercially available hydrophilic polymers linked, directly or via a spacer, to reactive chemistries that may be of use in the present invention include, but are not limited to, the following:

| | | Catalogue |
|---|---|---|
| PEG linker Description | Source of PEG | Number |
| TCR Monomer attachment | | |
| 5K linear (Maleimide) | Nektar | 2D2MOHO1 |
| 20K linear (Maleimide) | Nektar | 2D2MOPO1 |
| 20K linear (Maleimide) | NOF Corporation | SUNBRIGHT |
| | | ME-200MA |
| 20K branched (Maleimide) | NOF Corporation | SUNBRIGHT |
| | | GL2-200MA |
| 30K linear (Maleimide) | NOF Corporation | SUNBRIGHT |
| | | ME-300MA |
| 40K branched PEG (Maleimide) | Nektar | 2D3XOTO1 |
| 5K-NP linear (for Lys attachment) | NOF Corporation | SUNBRIGHT |
| | | MENP-50H |
| 10K-NP linear (for Lys attachment) | NOF Corporation | SUNBRIGHT |
| | | MENP-10T |
| 20K-NP linear (for Lys attachment) | NOF Corporation | SUNBRIGHT |
| | | MENP-20T |
| TCR dimer linkers | | |
| 3.4K linear (Maleimide) | Nektar | 2D2DOFO2 |
| 5K forked (Maleimide) | Nektar | 2D2DOHOF |
| 10K linear (with orthopyridyl ds- | Sunbio | |
| linkers in place of Maleimide) | | |
| 20K forked (Maleimide) | Nektar | 2D2DOPOF |
| 20K linear (Maleimide) | NOF Corporation | |
| 40K forked (Maleimide) | Nektar | 2D3XOTOF |
| Higher order TCR multimers | | |
| 15K, 3 arms, Mal₃ (for trimer) | Nektar | OJOONO3 |
| 20K, 4 arms, Mal₄(for tetramer) | Nektar | OJOOPO4 |
| 40K, 8 arms, Mal₈(for octamer) | Nektar | OJOOTO8 |

A wide variety of coupling chemistries can be used to couple polymer molecules to protein and peptide therapeutics. The choice of the most appropriate coupling chemistry is largely dependant on the desired coupling site. For example, the following coupling chemistries have been used attached to one or more of the termini of PEG molecules (Source: Nektar Molecular Engineering Catalogue 2003):

N-maleimide

Vinyl sulfone

Benzotriazole carbonate

Succinimidyl proprionate

Succinimidyl butanoate

Thio-ester

Acetaldehydes

Acrylates

Biotin

Primary amines

As stated above non-PEG based polymers also provide suitable linkers for multimerising the TCRs of the present invention. For example, moieties containing maleimide termini linked by aliphatic chains such as BMH and BMOE (Pierce, products Nos. 22330 and 22323) can be used.

Peptidic linkers are the other class of TCR linkers. These linkers are comprised of chains of amino acids, and function to produce simple linkers or multimerisation domains onto which TCR molecules can be attached. The biotin/streptavidin system has previously been used to produce TCR tetramers (see WO/99/60119) for in-vitro binding studies. However, stepavidin is a microbially-derived polypeptide and as such not ideally suited to use in a therapeutic.

There are a number of human proteins that contain a multimerisation domain that could be used in the production of multivalent TCR complexes. For example the tetramerisation domain of p53 which has been utilised to produce tetramers of scFv antibody fragments which exhibited increased serum persistence and significantly reduced off-rate compared to the monomeric scFV fragment. (Willuda et al. (2001) J. Biol. Chem. 276 (17) 14385-14392) Haemoglobin also has a tetramerisation domain that could potentially be used for this kind of application.

Cell Targeting with Multivalent TCR Complexes

The multivalent TCR complexes of the invention, particularly TCR dimers, have the advantage of exhibiting preferential association with target cells expressing the cognate TCR ligand for the TCR they incorporate. These complexes are also capable of penetrating tumour mass, most probably via the tumour blood supply. Example 10 herein provides experimental exemplification of the tumour penetrating and localising characteristics of an NY-ESO TCR dimer. These characteristics make the multivalent TCR complexes of the present invention suitable moieties for the delivery of therapeutic and/or imaging agents to cells expressing a given TCR ligand.

Therapeutic Use

The TCR complex of the present invention may be associated with (e.g. covalently or otherwise linked to) a therapeutic agent which may be, for example, a toxic moiety for use in cell killing, or an immunostimulating agent such as an interleukin, a cytokine, or an immunostimulatory peptide or polypeptide. Example immunostimulatory polypeptides include the NY-ESO polypeptide and the SLLMWITQC peptide loaded by HLA-A2 molecules on cancerous cells that contain the NY-ESO polypeptide, or the GILGFVFTL peptide loaded by HLA-A2 by cells infected with influenza. The said immuno-stimulatory polypeptides will preferably contain one or more peptide epitopes in a form than can be processed and presented by HLA molecules. Alternatively the immunostimulatory peptide may comprise a synthetic non-naturally occurring peptide, capable to being loaded by an HLA polypeptide. A recombinant TCR could then be produced that recognised the HLA-synthetic peptide complex for use in multivalent TCR complexes of the present invention. It is also contemplated that a plurality of said therapeutic agents might be associated with a multivalent TCR complex of the present invention.

A multivalent TCR complex of the present invention may have enhanced binding capability for a cognate ligand compared to a non-multimeric T cell receptor heterodimer. Thus, the multivalent TCR complexes according to the invention are particularly useful for tracking or targeting cells presenting particular antigens in vitro or in vivo, and are also useful as intermediates for the production of further multivalent TCR complexes having such uses. The TCR or multivalent TCR complex may therefore be provided in a pharmaceutically acceptable formulation for use in vivo.

The invention also provides a method for delivering a therapeutic agent to a target cell, which method comprises contacting potential target cells with a multivalent TCR complex in accordance with the invention under conditions to allow attachment of the multivalent TCR complex to the target cell, said multivalent TCR complex being specific for the cognate ligand (eg MHC-peptide complex, CD1-antigen complex, superantigen or MHC-peptide/superantigen complex) and having the therapeutic agent associated therewith.

It is currently believed that the therapeutic use of TCR complexes of the present invention, for example TCR-PEG dimers or such dimers linked to a therapeutic agent, offers additional, unexpected benefits. For example, distribution of such complexes is in many cases is largely confined to the viable areas of the tumours indicating that multimeric TCR complexes of the invention may be selectively targeted to this area of tumours. This is an important, and unexpected benefit, as it is these viable areas that a successful therapeutic must target and less of the complex may be required for a given effect if it is not wasted in targeting dead tumour cells. Furthermore, it is currently believed that such complexes are capable of rapid tumour penetration. Rapid internalisation indicates an active ingress mechanism. Without wishing to be bound by theory, it is postulated that this active mechanism may involve the internalisation of pMHC on tumour cells in response to interactions with TCR complexes, for example dimers. This internalisation may lead to the complexes associated with pMHC being “pulled into” the tumour cells. Furthermore, it has previously been suggested that internalisation of pMHC may lead to apoptosis, providing a further unexpected mode-of-action for such multivalent TCR complex therapeutics. This assertion is supported by a number of studies that demonstrate antigen presenting cells undergo apoptosis in response to the binding of anti-HLA antibodies. (See for example, (Wallen-Ohman et al., (1997) J. Immunology 9 (4) 599-606) and (Daniel et al., (2003) Transplantation 75 (8) 1380-6)). This observation provides a further basis for their use as effective anti-cancer agents, possibly without the need to link them to additional cytotoxic agents.

However, in a particular embodiment of this invention, the multivalent TCR complex can be used to deliver therapeutic agents to the location of cells presenting a particular antigen. This is useful in many situations and, in particular, against tumours. A therapeutic agent can be delivered such that it exercises its effect locally, but not only on the cell it binds to. Thus, one particular strategy uses anti-tumour molecules linked to multivalent TCR complexes of the invention specific for tumour antigens.

Many therapeutic agents may be employed for this use, for instance radioactive compounds, enzymes (perforin, for example) or chemotherapeutic agents (cisplatin, for example). To ensure that toxic effects are exercised in the desired location the toxin may be inside a liposome linked to streptavidin so that the compound is released slowly. This prevents damaging effects during the transport in the body and ensures that the toxin has maximum effect after binding of the multivalent TCR complex to the relevant antigen presenting cells.

Other suitable therapeutic agents include:
small molecule cytotoxic agents, i.e. compounds with the ability to kill mammalian cells having a molecular weight of less than 700 daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolmide, topotecan, trimetreate glucuronate, auristatin E vincristine and doxorubicin;
peptide cytotoxins, i.e. proteins or fragments thereof with the ability to kill mammalian cells. Examples include ricin, diphtheria toxin, pseudomonas bacterial exotoxin A, DNAase and RNAase;
radio-nuclides, i.e. unstable isotopes of elements which decay with the concurrent emission of one or more of α or β particles, or γ rays. Examples include iodine 131, rhenium 186, indium 111, yttrium 90 and complexes incorporating ⁹⁰Yt such as ⁹⁰Yt-DOTA-Biotin, bismuth 210 and 213, actinium 225 and astatine 213;
prodrugs, such as antibody directed enzyme pro-drugs;
immuno-stimulants, i.e. moieties which stimulate immune response. Examples include cytokines such as IL-2, chemokines such as IL-8, platelet factor 4, melanoma growth stimulatory protein, etc, antibodies or fragments thereof, complement activators, xenogeneic protein domains, allogeneic protein domains, viral/bacterial protein domains and viral/bacterial peptides.

Soluble multivalent TCR complexes of the invention may be linked to an enzyme capable of converting a prodrug to a drug. This allows the prodrug to be converted to the drug only at the site where it is required (i.e. targeted by the sTCR).

Examples of suitable MHC-peptide targets for the TCR according to the invention include, but are not limited to, viral epitopes such as HTLV-1 epitopes (e.g. the Tax peptide restricted by HLA-A2; HTLV-1 is associated with leukaemia), HIV epitopes, EBV epitopes, CMV epitopes; melanoma epitopes (e.g. MAGE-1 HLA-A1 restricted epitope) and other cancer-specific epitopes (e.g. the renal cell carcinoma associated antigen G250 restricted by HLA-A2); and epitopes associated with autoimmune disorders, such as rheumatoid arthritis. Further disease-associated pMHC targets, suitable for use in the present invention, are listed in the HLA Factbook (Barclay (Ed) Academic Press), and many others are being identified.

A multitude of disease treatments can potentially be enhanced by localising the drug through the specificity of soluble TCRs.

Viral diseases for which drugs exist, e.g. HIV, SIV, EBV, CMV, would benefit from the drug being released or activated in the near vicinity of infected cells. For cancer, the localisation in the vicinity of tumours or metastasis would enhance the effect of toxins or immunostimulants. In autoimmune diseases, immunosuppressive drugs could be released slowly, having more local effect over a longer time-span while minimally affecting the overall immuno-capacity of the subject. In the prevention of graft rejection, the effect of immunosuppressive drugs could be optimised in the same way. For vaccine delivery, the vaccine antigen could be localised in the vicinity of antigen presenting cells, thus enhancing the efficacy of the antigen. The method can also be applied for imaging purposes.

The soluble multivalent TCR complexes of the present invention may be used to modulate T cell activation by binding to specific cognate ligands and thereby inhibiting T cell activation. Autoimmune diseases involving T cell-mediated inflammation and/or tissue damage would be amenable to this approach, for example type I diabetes. Knowledge of the specific peptide epitope presented by the relevant pMHC is required for this use. An alternative means of treating autoimmune disease is to use multivalent TCR complexes of the present invention that comprise TCRs capable of binding to HLA molecules of a given type loaded with a wide range of, or any, suitable peptide. The use of such multivalent TCR complexes is expected to lead to the suppression of T cell responses mediated by interaction with complexes comprising said HLA molecule. It is well known that many autoimmune diseases are associated with a particular HLA type or types and these associations can be used to select the appropriate HLA that a multimeric TCR complex of the present invention should recognise. For example, Rheumatoid Arthritis is associated with HLA-DR4 and Ankylosing Spondylitis is associated with HLA-B27. A discussion of the association between HLA types and many diseases can be found in (Lechler, (2000) HLA in Health and Disease, 2nd Edition, Academic Press).

The TCR complexes of the present invention may be used to modulate T cell activation by binding to specific TCR ligand and thereby inhibiting T cell activation. Autoimmune diseases involving T cell-mediated inflammation and/or tissue damage would be amenable to this approach, for example type I diabetes. Knowledge of the specific peptide epitope presented by the relevant pMHC is required for this use.

Therapeutic or imaging TCR complexes in accordance with the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example parenteral, transdermal or via inhalation, preferably a parenteral (including subcutaneous, intramuscular, or, most preferably intravenous) route. Such compositions may be prepared by any method known in the art of pharmacy, for example by admixing the active ingredient with the carrier(s) or excipient(s) under sterile conditions.

Dosages of the substances of the present invention can vary between wide limits, depending upon the disease or disorder to be treated, the age and condition of the individual to be treated, etc. and a physician will ultimately determine appropriate dosages to be used.

Gene cloning techniques may be used to provide TCRs for conjugation as complexes of the invention. These techniques are disclosed, for example, in J. Sambrook et al Molecular Cloning 2nd Edition, Cold Spring Harbor Laboratory Press (1989).

Diagnostic Uses

As stated above the multivalent TCR complexes of the present invention have applications relating to the imaging, tracking and targeting of cells and cell masses. Moieties that facilitate imaging of these cells can be associated with these complexes. Such labelled multivalent TCR complexes can be used to analyse the distribution of cells expressing the cognate TCR ligand for the TCR incorporated within a given multivalent TCR complex. This imaging can be carried out either in-vivo or ex-vivo. There is a range of imaging agents, known to those skilled in the art, which could be associated with the multivalent TCR complexes of the present invention. These imaging agents include, but are not limited to, the following:

Radionuclides (e.g. ¹²⁵I, ²⁰¹Tl, ⁶⁷Ga, ¹⁷F, ¹³¹ I and ^{99m}Tc,)

Electro-dense particles (e.g. gold)

Fluorescent labels (e.g. FITC, PE, CY-3 and CY-5)

Multivalent TCR complexes associated with such labelling moieties are useful in methods for the diagnosis of cancer and infectious diseases, as well as for monitoring progression of the disease or cancer.

Additional Aspects

The soluble multivalent TCRs present in complexes of the present invention may obtained by expression in a bacterium such as E. coli as inclusion bodies, and subsequent refolding in vitro.

Refolding of the TCR chains may take place in vitro under suitable refolding conditions. In a particular embodiment, a TCR with correct conformation is achieved by refolding solubilised TCR chains in a refolding buffer comprising a solubilising agent, for example urea. Advantageously, the urea may be present at a concentration of at least 0.1 M or at least 1 M or at least 2.5 M, or about 5 M. An alternative solubilising agent which may be used is guanidine, at a concentration of between 0.1 M and 8 M, preferably at least 1 M or at least 2.5 M. Prior to refolding, a reducing agent is preferably employed to ensure complete reduction of cysteine residues. Further denaturing agents such as DTT and guanidine may be used as necessary. Different denaturants and reducing agents may be used prior to the refolding step (e.g. urea, β-mercaptoethanol). Alternative redox couples may be used during refolding, such as a cystamine/cysteamine redox couple, DTT or β-mercaptoethanol/atmospheric oxygen, and cysteine in reduced and oxidised forms.

Folding efficiency may also be increased by the addition of certain other protein components, for example chaperone proteins, to the refolding mixture. Improved refolding has been achieved by passing protein through columns with immobilised mini-chaperones (Altamirano, et al. (1999). Nature Biotechnology 17: 187-191; Altamirano, et al. (1997). Proc Natl Acad Sci USA 94(8): 3576-8).

Alternatively, multivalent TCR complexes of the present invention may obtained by expression in a eukaryotic cell system, such as insect cells.

Purification of the multivalent TCR complexes may be achieved by many different means. Alternative modes of ion exchange may be employed or other modes of protein purification may be used such as gel filtration chromatography or affinity chromatography.

Preferred features of each aspect of the invention are as for each of the other aspects mutatis mutandis. The prior art documents mentioned herein are incorporated to the fullest extent permitted by law.

### EXAMPLES

The invention is further described in the following examples, which do not limit the scope of the invention in any way.

Reference is made in the following to the accompanying drawings in which:

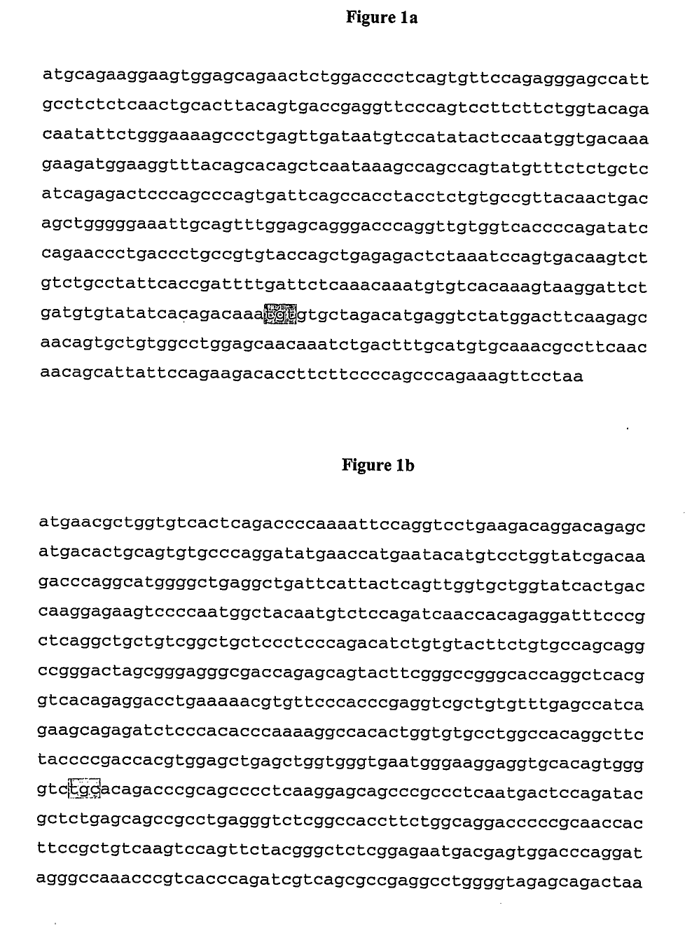

FIGS. 1a and 1b show respectively the nucleic acid sequences of the α and β chains of a soluble A6 TCR, mutated so as to introduce a cysteine codon. The shading indicates the introduced cysteine codons;

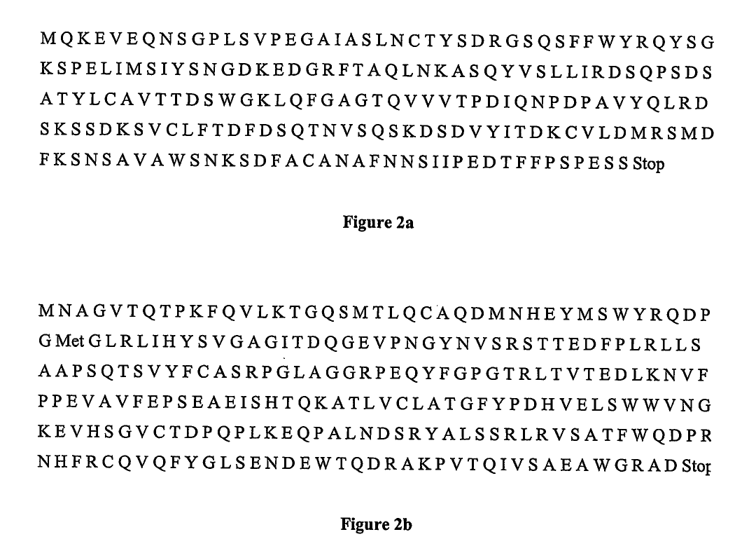

FIG. 2a shows the A6 TCR α chain extracellular amino acid sequence, including the T₄₈→C mutation (underlined) used to produce the novel disulphide inter-chain bond, and FIG. 2b shows the A6 TCR β chain extracellular amino acid sequence, including the S₅₇→C mutation (underlined) used to produce the novel disulphide inter-chain bond;

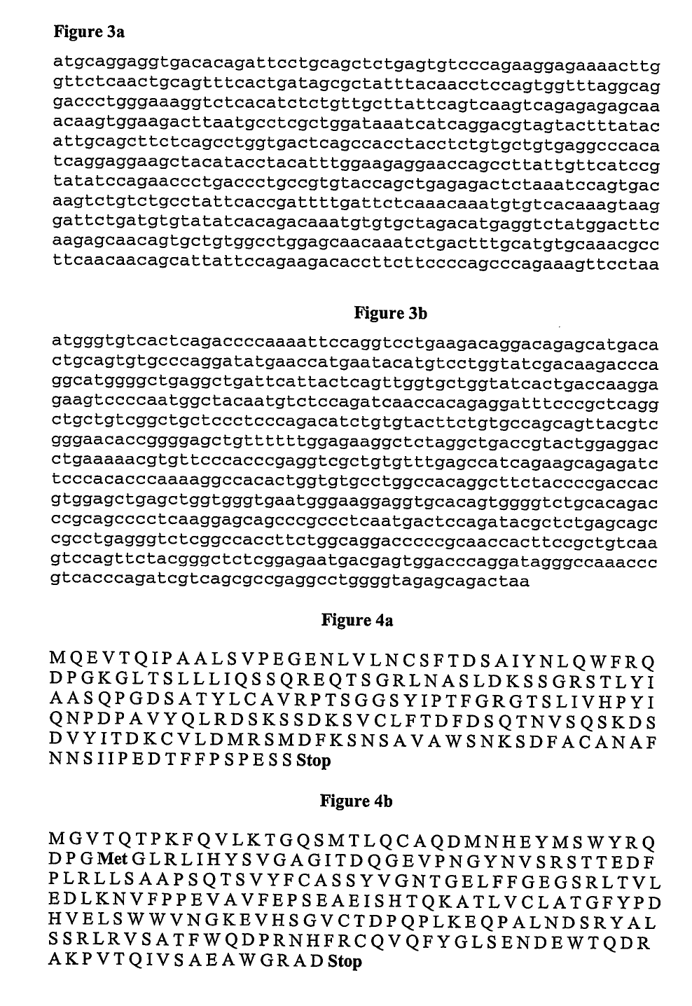

FIGS. 3a and 3b show respectively the nucleic acid sequences of the α and β chains of a soluble NY-ESO TCR, mutated so as to introduce a cysteine codon.

FIG. 4a shows the NY-ESO TCR α chain extracellular amino acid sequence, including the T₄₈→C mutation used to produce the novel disulphide inter-chain bond, and FIG. 4b shows the A6 TCR β chain extracellular amino acid sequence, including the S₅₇→C mutation used to produce the novel disulphide inter-chain bond;

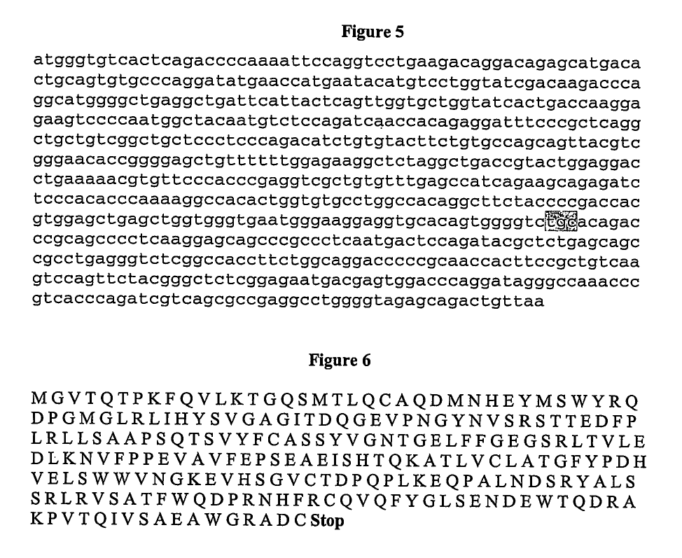

FIG. 5 shows the nucleic acid sequence of the β chain of a soluble NY-ESO TCR, further mutated so as to introduce codon causing the addition of a cysteine residue on the C-terminus of the encoded polypeptide.

FIG. 6 shows the amino acid sequence of the β chain of a soluble NY-ESO TCR, further mutated so as to introduce a cysteine residue on the C-terminus of the polypeptide.

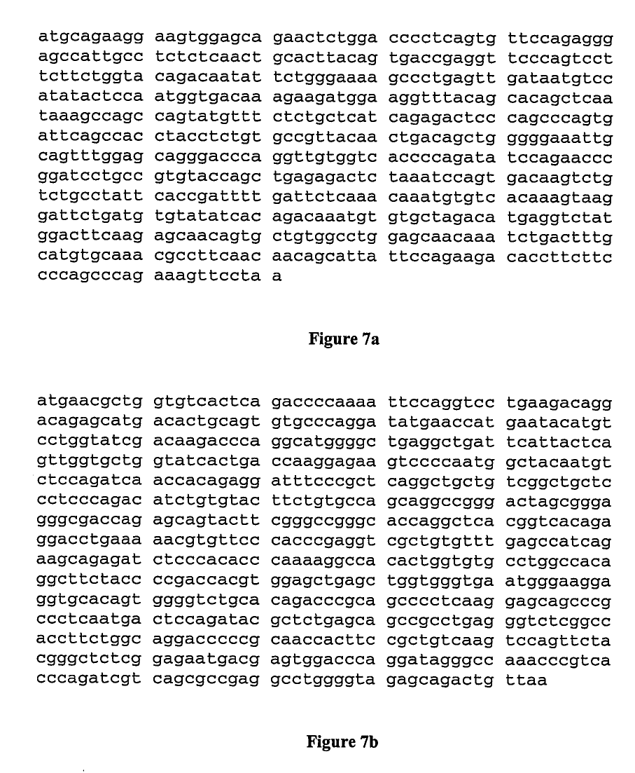

FIGS. 7a and 7b show respectively the nucleic acid sequences of the α and β chains of a soluble A6 TCR, further mutated so as to introduce codon causing the addition of a cysteine residue on the C-terminus of the encoded polypeptide.

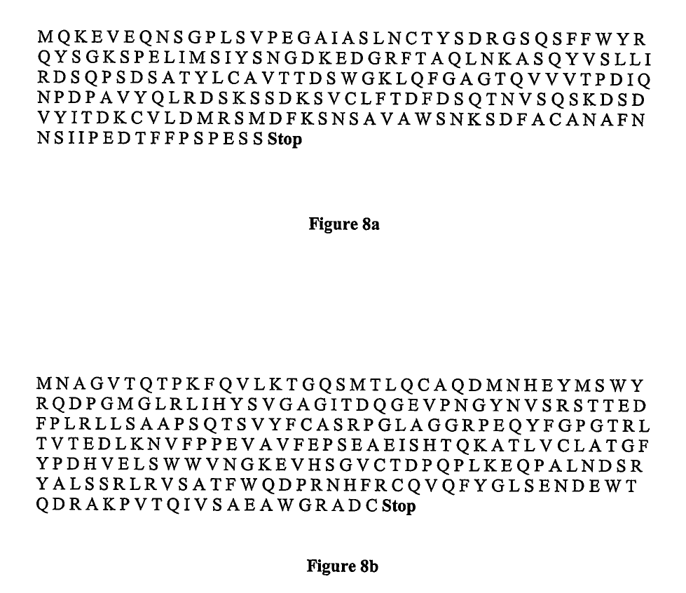

FIGS. 8a and 8b show respectively the amino acid sequences of the α and β chains of a soluble A6 TCR, further mutated so as to introduce a cysteine residue on the C-terminus of the polypeptide.

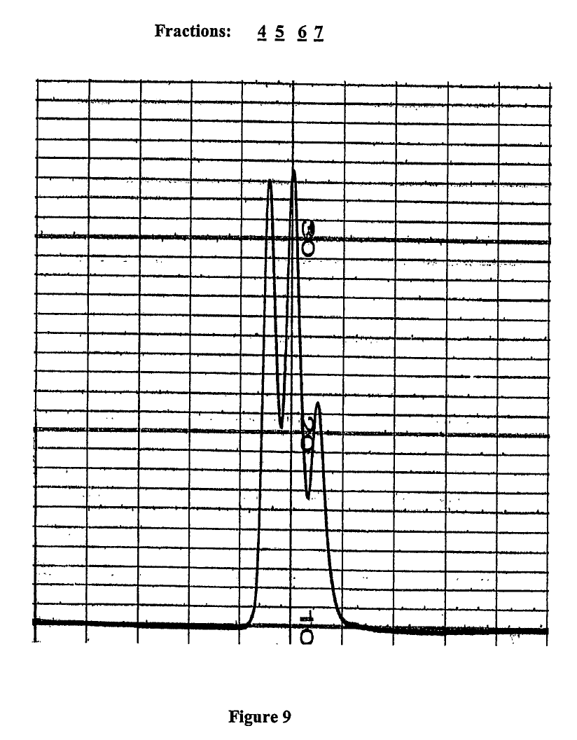

FIG. 9 is a chromatogram of an dimeric NY-ESO TCR 3.4 kd Mal-PEG-Mal complex run on a Superdex 75 HR10/30 gel-filtration column pre-equilibrated in PBS.

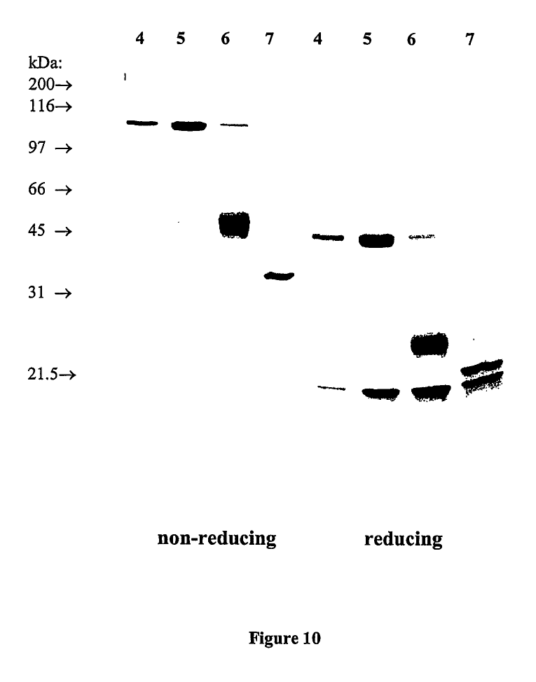

FIG. 10 is an SDS PAGE gel of fractions collected from the gel-filtration gel illustrated in FIG. 9 run under reducing and non-reducing conditions.

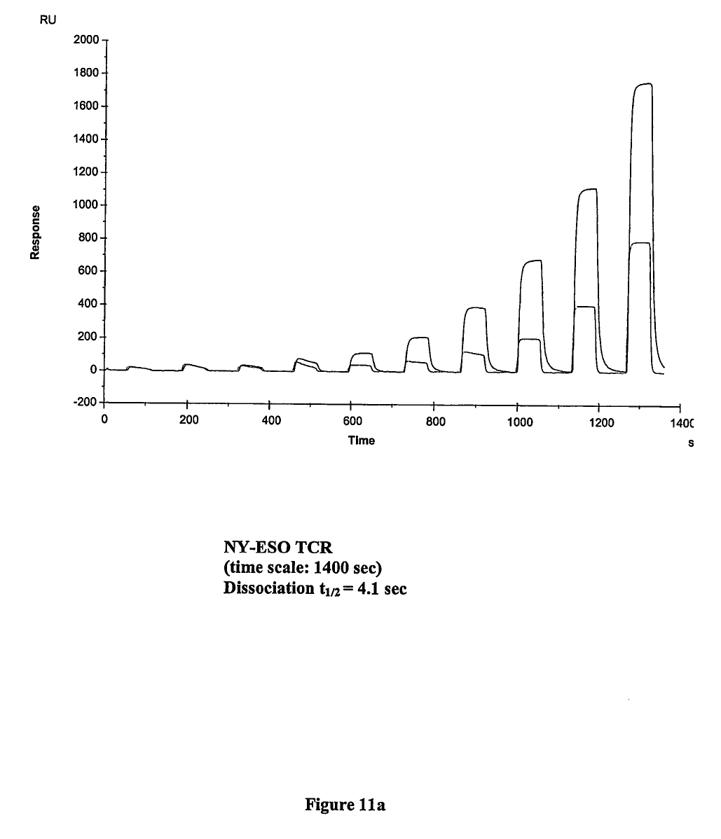

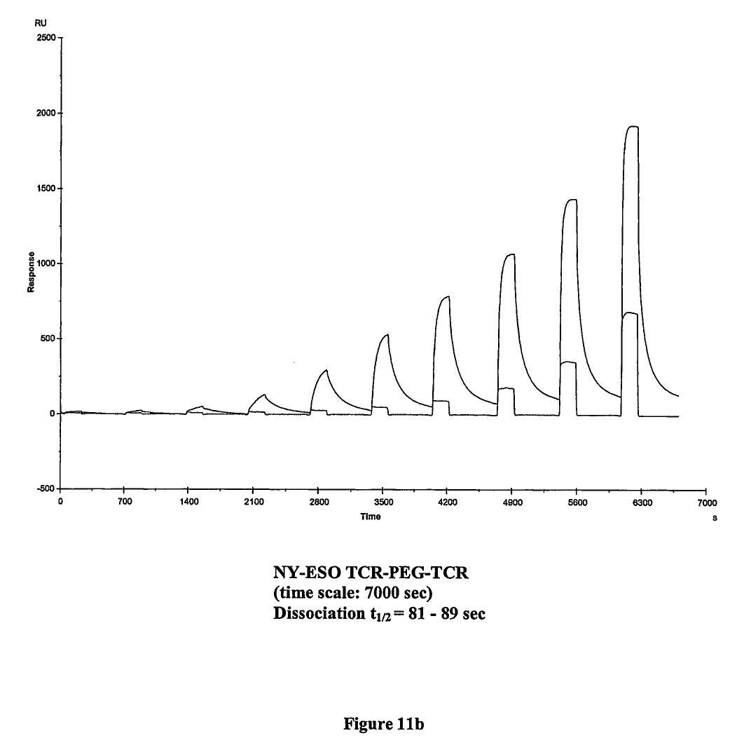

FIG. 11a is a BIACore trace that shows the interaction between monomeric NY-ESO TCR and immobilised HLA-A2-NY-ESO.

FIG. 11b is a BIACore trace that shows the interaction between a dimeric NY-ESO TCR 3.4 kd Mal-PEG-Mal complex and immobilised HLA-A2-NY-ESO.

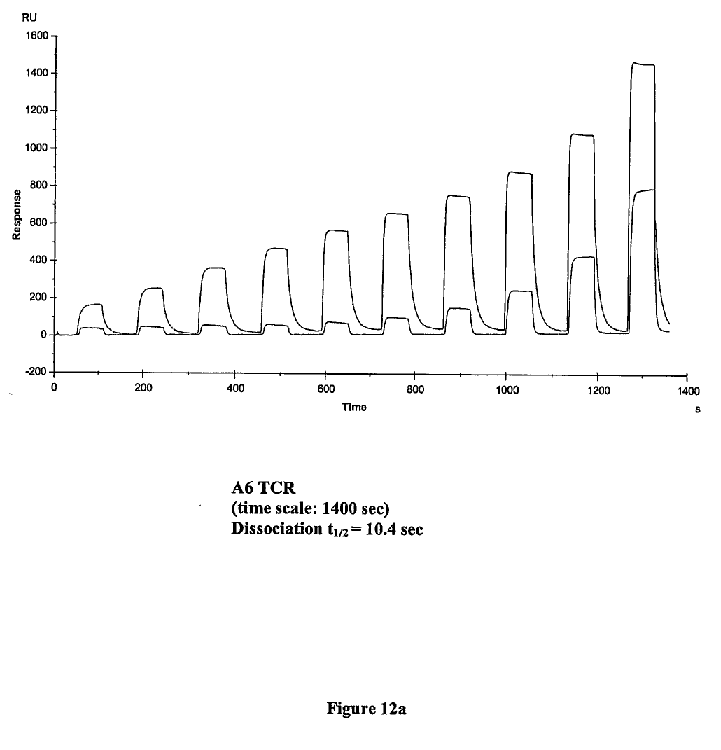

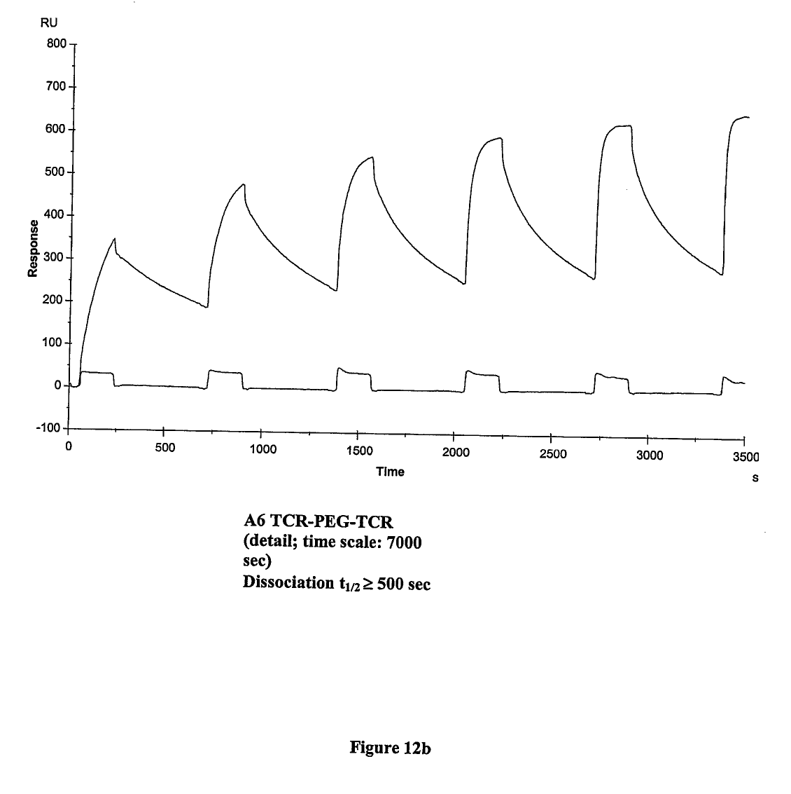

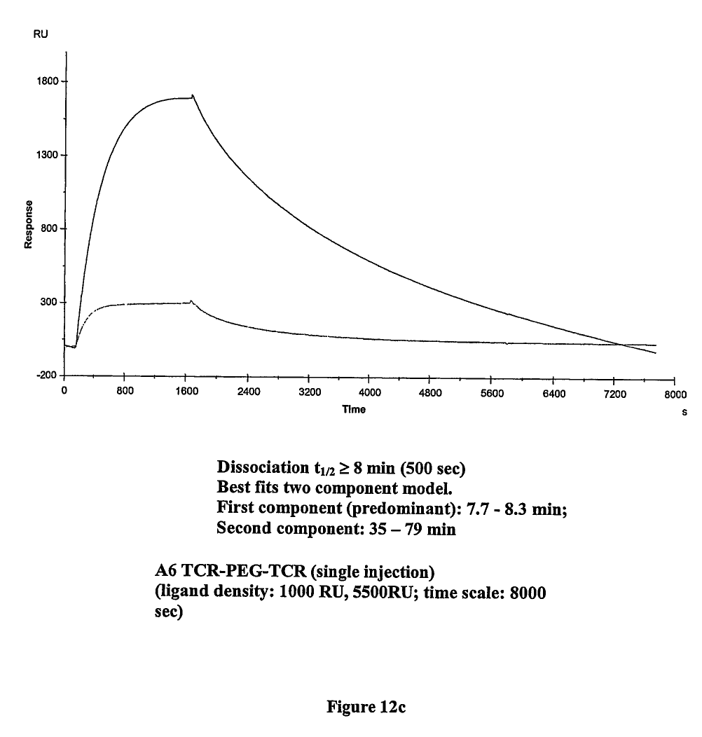

FIG. 12a is a BIACore trace that shows the interaction between monomeric A6 TCR and immobilised HLA-A2-Tax.

FIG. 12b is a BIACore trace that shows the interaction between a dimeric A6 TCR 3.4 kd Mal-PEG-Mal complex and immobilised HLA-A2-Tax

FIG. 12c is a BIACore trace that shows a single injection of a dimeric A6 TCR 3.4 kd Mal-PEG-Mal complex flowed over immobilised HLA-A2-Tax.

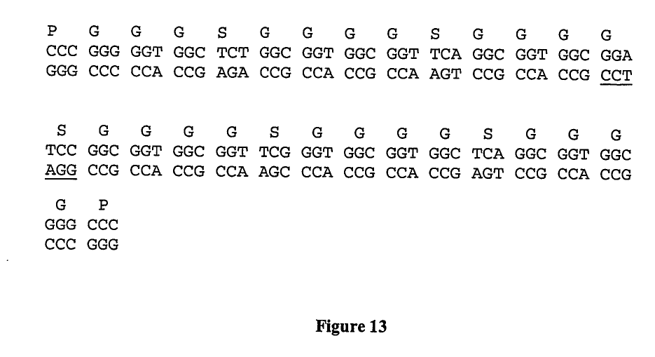

FIG. 13 shows the DNA and amino acid sequences of the linker used in the construction of the A6 scTCR.

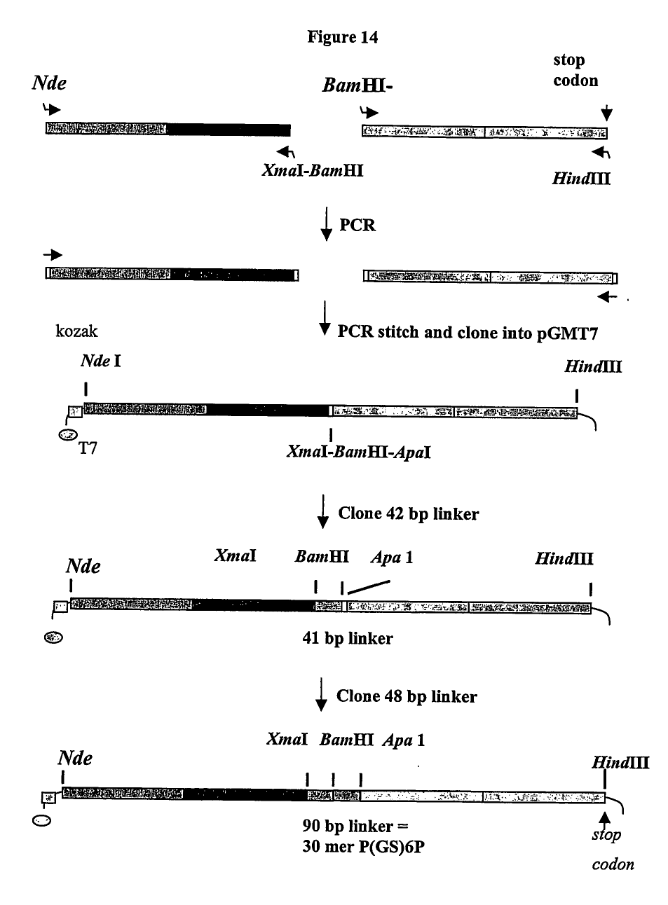

FIG. 14 Outlines the cloning of TCR α and β chains into phagmid vectors. The diagram describes a phage display vector. RSB is the ribosome-binding site. S1 or S2 are signal peptides for secretion of proteins into periplasm of E. coli. The * indicates translation stop codon. Either of the TCR α chain or β chain can be fused to phage coat protein, however in this diagram only TCR β chain is fused to phage coat protein.

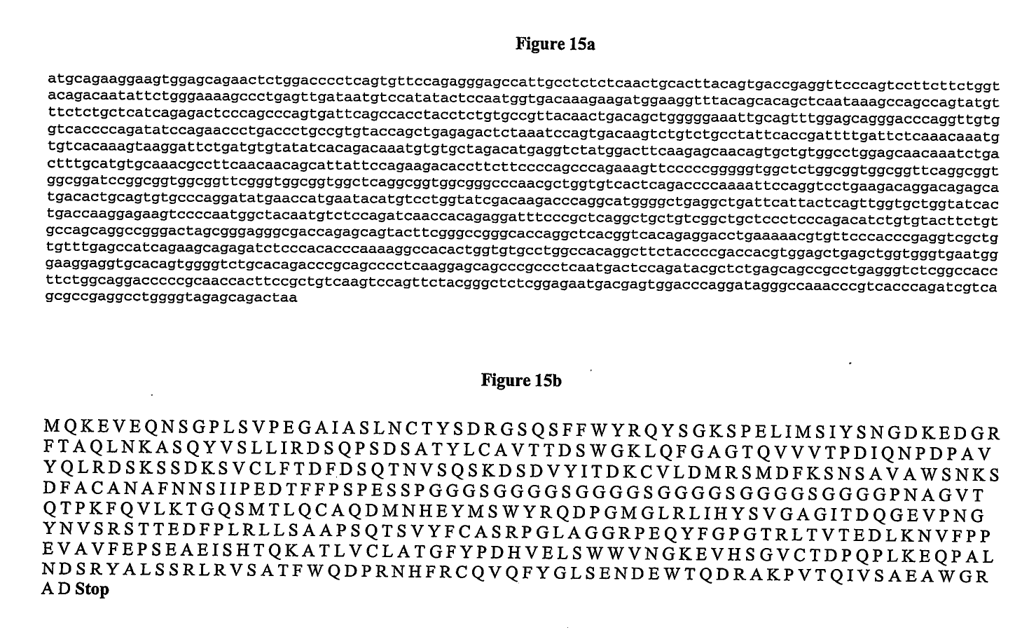

FIGS. 15a and 15b detail the DNA and amino acid sequence of phagmid pEX746:A6 respectively.

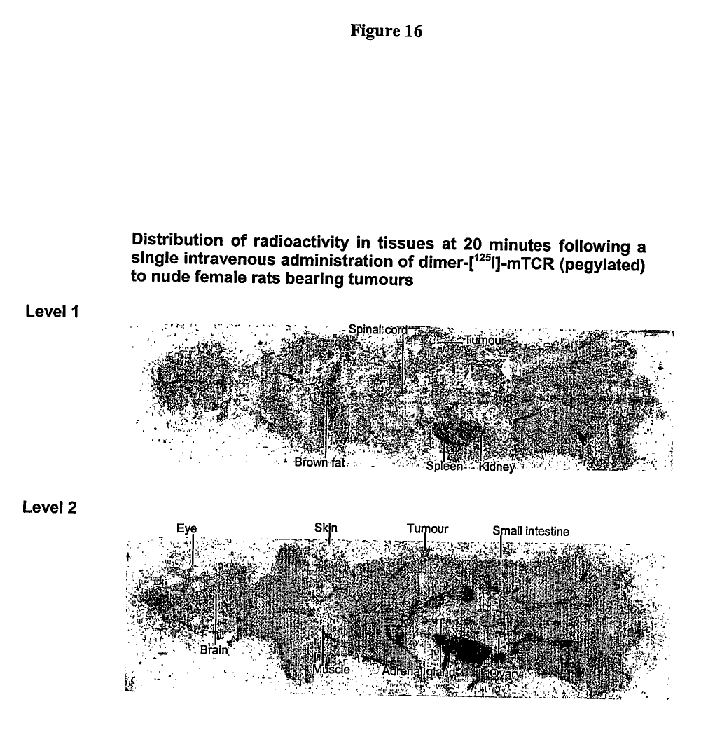

FIG. 16 illustrates the distribution of radioactivity in tissues at 20 minutes following a single intravenous administration of dimer-[¹²⁵I]-mTCR (pegylated) to nude female rats bearing tumours.

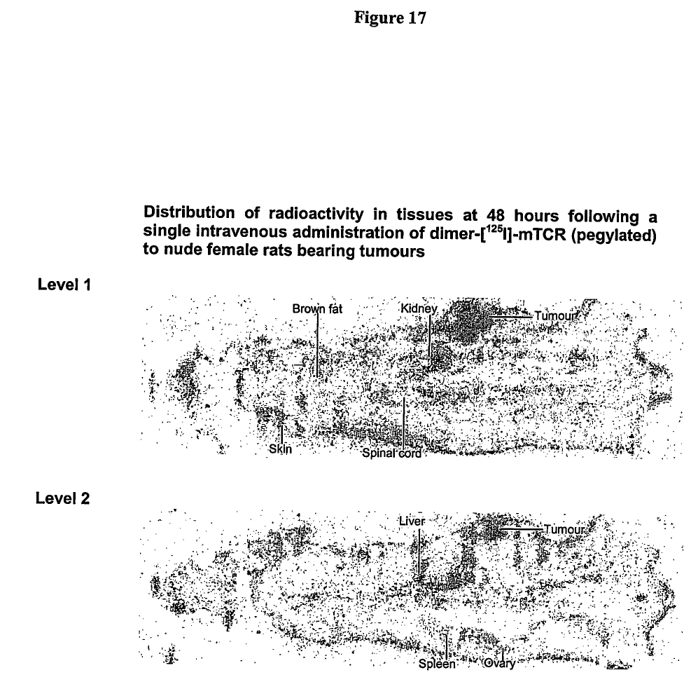

FIG. 17 illustrates the distribution of radioactivity in tissues at 48 hours following a single intravenous administration of dimer-[¹²⁵ I]-mTCR (pegylated) to nude female rats bearing tumours.

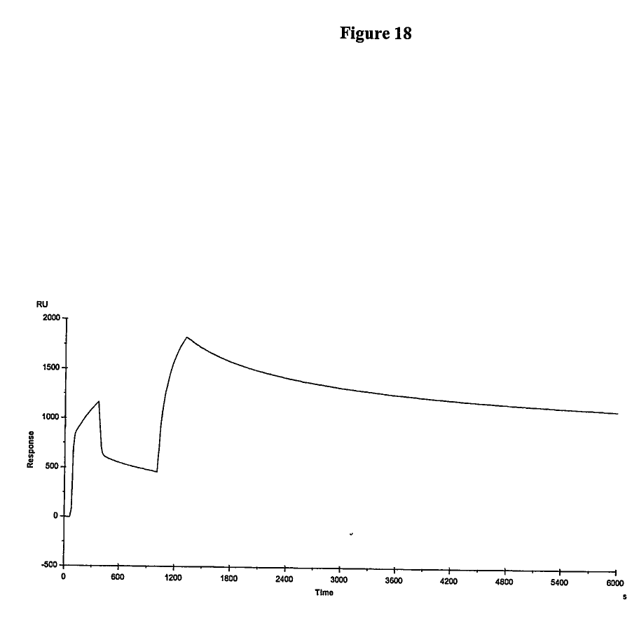

FIG. 18 is a BIAcore trace that shows the interaction between a PEG-linked A6 TCR tetramer and immobilised HLA-A2-Tax.

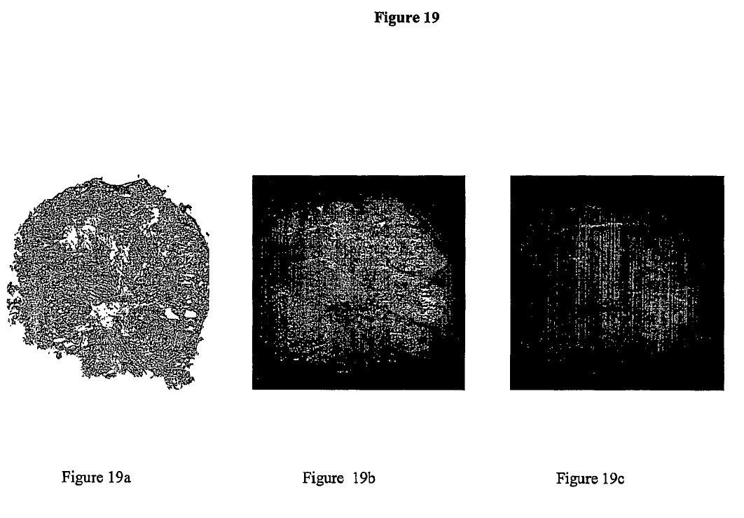

FIG. 19a illustrates an H&E stained cryostat tumour section.

FIG. 19b illustrates an anti-HLA-A2 stained cryostat tumour section.

FIG. 19c illustrates a control IgG stained cryostat tumour section.

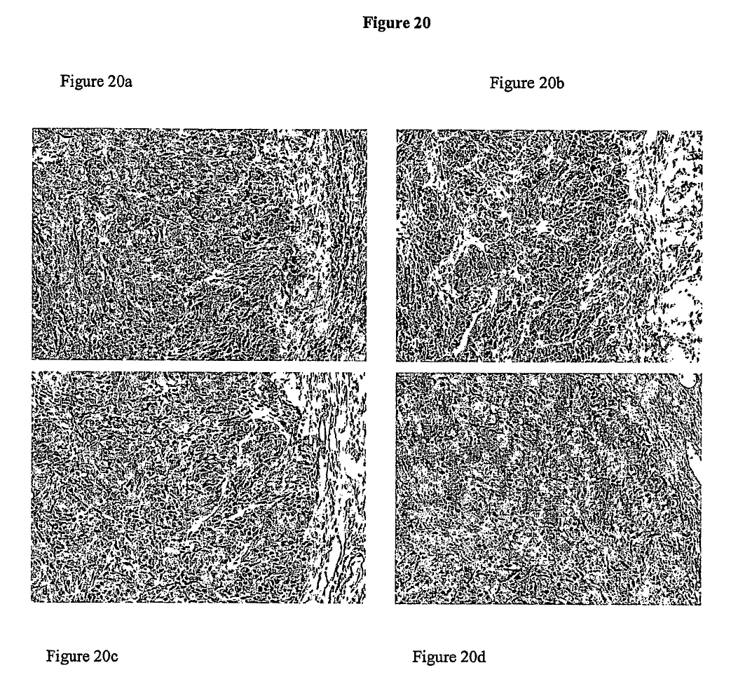

FIG. 20a illustrates an H&E and anti-TCR β chain antibody stained formalin-fixed paraffin-embedded tumour section.

FIG. 20b illustrates an H&E and anti-NY-ESO TCR antibody stained formalin-fixed paraffin-embedded tumour section.

FIG. 20c illustrates an H&E and anti-NY-ESO antibody/NY-ESO TCR control stained formalin-fixed paraffin-embedded tumour section.

FIG. 20d illustrates an H&E and omission control stained formalin-fixed paraffin-embedded tumour section.

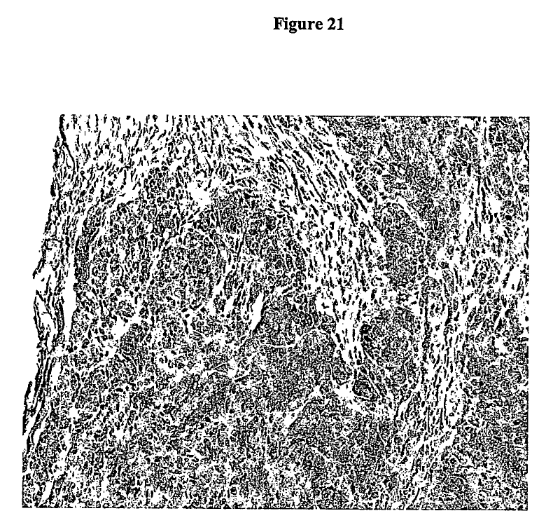

FIG. 21 illustrates an H&E and anti-NY-ESO TCR antibody stained formalin-fixed paraffin-embedded tumour section.

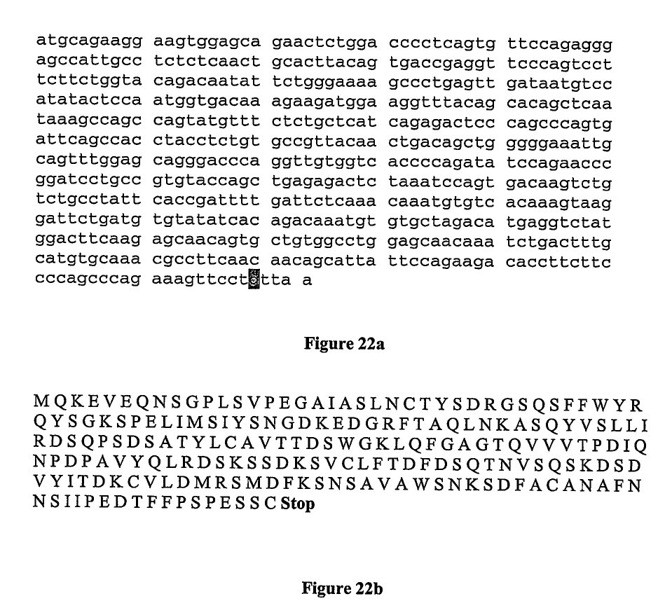

FIG. 22a details the DNA sequence of the high affinity A6 TCR α chain; including the introduced cysteine codon at the 3′ end.

FIG. 22b details the amino acid sequence of the A6 TCR α chain; including the introduced cysteine codon at the 3′ end.

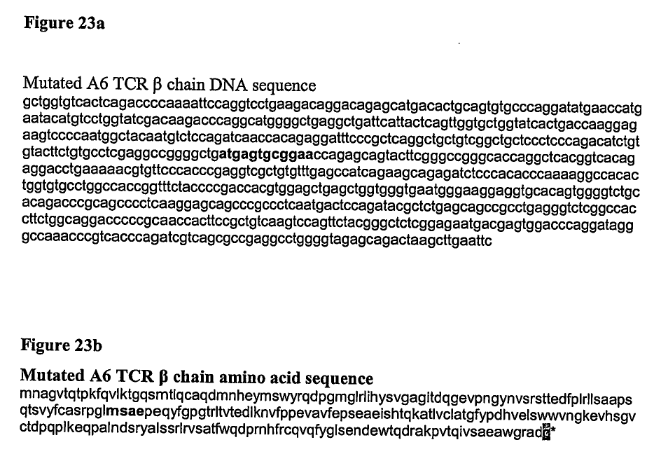

FIG. 23a details the DNA sequence of the high affinity A6 TCR β chain; the mutated nucleic acids are indicated in bold.

FIG. 23b details the amino acid sequence of the A6 TCR β chain; the mutated amino acids are indicated in bold.

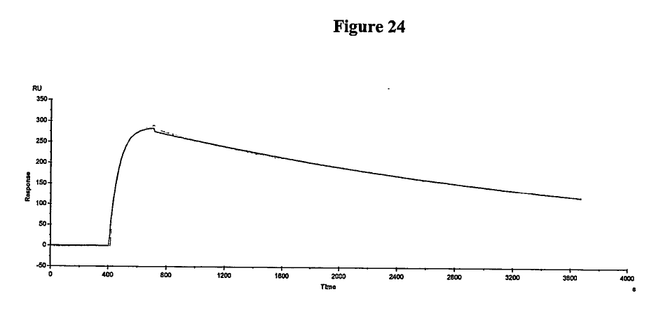

FIG. 24 is a BIAcore trace that shows the interaction between a high affinity A6 TCR and immobilised HLA-A2-Tax.

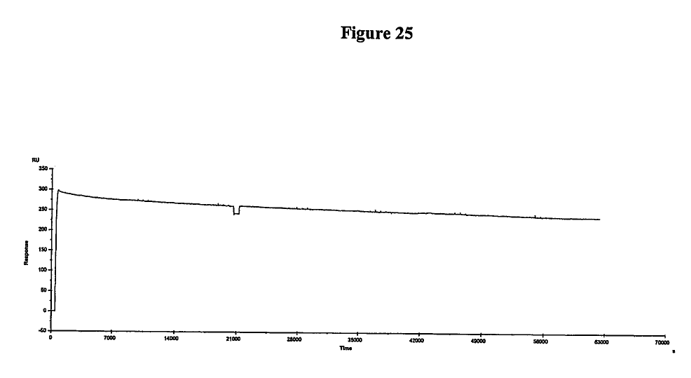

FIG. 25 is a BIAcore trace that shows the interaction between a divalent high affinity A6 TCR 3.4 KD Mal-PEG-Mal complex and immobilised HLA-A2-Tax.

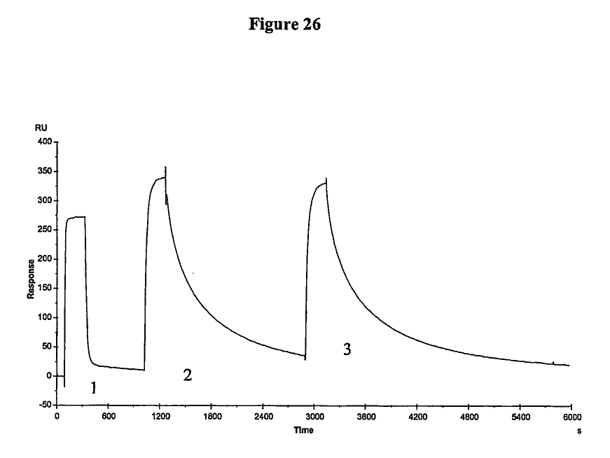

FIG. 26 is a BIAcore trace that shows the interaction between A6 TCR PEG complexes and immobilised HLA-A2-Tax:

Peak 1—monomeric 3.4 KD A6 TCR PEG complexes

Peak 2—dimeric 3.4 KD A6 TCR linear Mal-PEG-Mal complexes

Peak 3—dimeric 5 KD A6 TCR forked Mal-PEG-Mal complexes

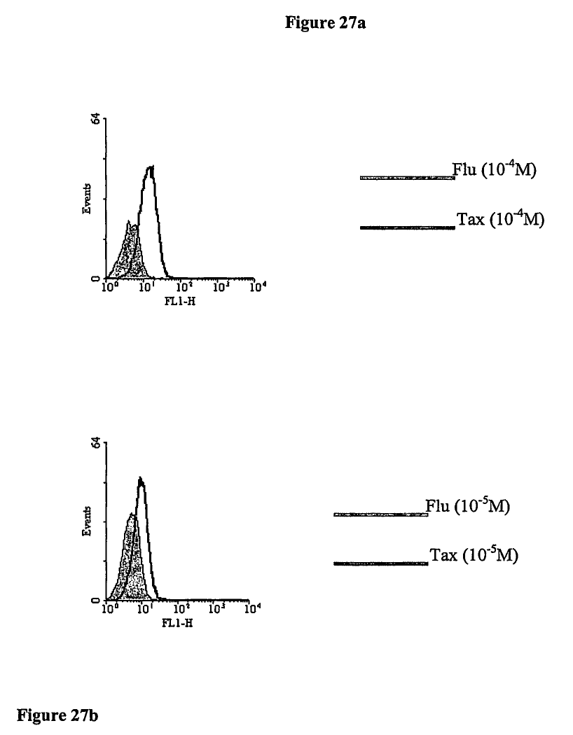

FIG. 27a illustrates the specific staining of PP cells pulsed with Tax peptide at 10⁻⁴ M by high affinity clone 134 A6 TCR 20 KD PEG dimers

FIG. 27b illustrates the specific staining of PP cells pulsed with Tax peptide at 10⁻⁵ M by high affinity clone 134 A6 TCR 20 KD PEG dimers

### Example 1

### Design of Primers and Mutagenesis of A6 Tax TCR α and β Chains

For mutating A6 Tax threonine 48 of exon 1 in TRAC*01 to cysteine, the following primers were designed (mutation shown in lower case):

5′-C ACA GAC AAA tgT GTG CTA GAC AT
5′-AT GTC TAG CAC Aca TTT GTC TGT G

For mutating A6 Tax serine 57 of exon 1 in both TRBC1*01 and TRBC2*01 to cysteine, the following primers were designed (mutation shown in lower case):

5′-C AGT GGG GTC tGC ACA GAG CC
5′-GG GTC TGT GCa GAC CCC ACT G

PCR Mutagenesis:

Expression plasmids containing the genes for the A6 Tax TCR α or β chain were mutated using the α-chain primers or the β-chain primers respectively, as follows. 100 ng of plasmid was mixed with 5 μl 10 mM dNTP, 25 μl 10×Pfu-buffer (Stratagene), 10 units Pfu polymerase (Stratagene) and the final volume was adjusted to 240 μl with H₂O. 48 μl of this mix was supplemented with primers diluted to give a final concentration of 0.2 μM in 50 μl final reaction volume. After an initial denaturation step of 30 seconds at 95° C., the reaction mixture was subjected to 15 rounds of denaturation (95° C., 30 sec.), annealing (55° C., 60 sec.), and elongation (73° C., 8 min.) in a Hybaid PCR express PCR machine. The product was then digested for 5 hours at 37° C. with 10 units of DpnI restriction enzyme (New England Biolabs). 10 μl of the digested reaction was transformed into competent XL1-Blue bacteria and grown for 18 hours at 37° C. A single colony was picked and grown over night in 5 ml TYP+ampicillin (16 g/l Bacto-Tryptone, 16 g/l Yeast Extract, 5 g/l NaCl, 2.5 g/l K₂HPO₄, 100 mg/l Ampicillin). Plasmid DNA was purified on a Qiagen mini-prep column according to the manufacturer's instructions and the sequence was verified by automated sequencing at the sequencing facility of Department of Biochemistry, Oxford University. The respective mutated nucleic acid and amino acid sequences are shown in FIGS. 1a and 2a for the α chain and FIGS. 1b and 2b for the β chain.

### Example 2

### Production of Soluble NY-ESO TCR Containing a Novel Disulphide Bond

cDNA encoding NY-ESO TCR was isolated from T cells supplied by Enzo Cerundolo (Institute of Molecular Medicine, University of Oxford) according to known techniques. cDNA encoding NY-ESO TCR was produced by treatment of the mRNA with reverse transcriptase.

The β chain of the soluble A6 TCR prepared in Example 1 contains in the native sequence a BglII restriction site (AAGCTT) suitable for use as a ligation site.

PCR mutagenesis was carried as detailed below to introduce a BamH1 restriction site (GGATCC) into the α chain of soluble A6 TCR, 5′ of the novel cysteine codon. The sequence described in FIG. 1a was used as a template for this mutagenesis. The following primers were used:

|BamHI |
5′-ATATCCAGAACCCgGAtCCTGCCGTGTA-3′
5′-TACACGGCAGGAaTCcGGGTTCTGGATAT-3′

In order to produce a soluble NY-ESO TCR incorporating a novel disulphide bond, A6 TCR plasmids containing the α chain BamHI and β chain BglII restriction sites were used as templates. The following primers were used:

```
                        | NdeI |
5′-GGAGATATACATATGCAGGAGGTGACACAG-3′
5′-TACACGGCAGGATCCGGGTTCTGGATATT-3′
                        | BamHI |
                        |NdeI |
5′-GGAGATATACATATGGGTGTCACTCAGACC-3′
5′-CCCAAGCTTAGTCTGCTCTACCCCAGGCCTCGGC-3′
          |BglII |
```

NY-ESO TCR α and α-chain constructs were obtained by PCR cloning as follows. PCR reactions were performed using the primers as shown above, and templates containing the NY-ESO TCR chains. The PCR products were restriction digested with the relevant restriction enzymes, and cloned into pGMT7 to obtain expression plasmids. The sequence of the plasmid inserts were confirmed by automated DNA sequencing. FIGS. 3a and 3b show the DNA sequence of the mutated α and β chains of the NY-ESO TCR respectively, and FIGS. 4a and 4b show the resulting amino acid sequences.

### Example 3

### Production of Soluble NY-ESO TCR Containing a Novel Disulphide Inter-Chain Bond, and an Additional Cysteine Residue on the C-Terminus of the β-Chain

In order to produce a soluble NY-ESO TCR incorporating a novel disulphide bond and a cysteine residue on the C-terminus of the β chain plasmids containing the α chain BamHI and β chain BglII restriction sites were used as a framework as described in Example 2. The following primers were used:

```
                        | NdeI |
5′-GGAGATATACATATGCAGGAGGTGACACAG-3′
5′-TACACGGCAGGATCCGGGTTCTGGATATT-3′
                        | BamHI |
                        | NdeI |
5′-GGAGATATACATATGGGTGTCACTCAGACC-3′
5′-CCCAAGCTTAACAGTCTGCTCTACCCCAGGCCTCGGC-3′
          |BglII |
```

NY-ESO TCR α and β-chain constructs were obtained by PCR cloning as follows. PCR reactions were performed using the primers as shown above, and templates containing the NY-ESO TCR chains. The PCR products were restriction digested with the relevant restriction enzymes, and cloned into pGMT7 to obtain expression plasmids. The sequence of the plasmid inserts were confirmed by automated DNA sequencing. FIG. 5 shows the DNA sequence of the mutated β chains of the NY-ESO TCR, and FIG. 6 shows the resulting amino acid sequence.

### Example 4

### Production of Soluble A6 TCR Containing a Novel Disulphide Inter-Chain Bond, and an Additional Cysteine Residue on the C-Terminus of the β-Chain

Plasmids encoding the A6 TCR containing the α chain BamHI and β chain BglII restriction sites, prepared as described in Example 2 were used as a starting point. The following primers were used to produce a soluble A6 TCR incorporating a novel disulphide bond and a cysteine residue on the C-terminus of the β chain as described in Example 3:

5′ - GGAGATATACATATGAACGCTGGTGTCACT - 3′
5′ - CCCAAGCTTAACAGTCTGCTCTACCCCAGGCCTCGGC - 3′

The sequence of the plasmid inserts were confirmed by automated DNA sequencing. FIGS. 7a and 8a show the DNA and amino acid sequence of the α chain of the mutated A6 TCR, and FIGS. 7b and 8b show the DNA and amino acid sequence of the β chain of the mutated A6 TCR

### Example 5

### Expression and Refolding of Soluble A6 and NY-ESO TCRs Containing a Novel Disulphide Inter-Chain Bond, and an Additional Cysteine Residue on the C-Terminus of the β-Chain

The expression plasmids detailed in Examples 3 and 4 encoding the mutated NY-ESO and A6 TCRs containing a novel disulphide inter-chain bond, and an additional cysteine residue on the C-terminus of the β-chains were transformed separately into E. coli strain BL21pLysS, and single ampicillin-resistant colonies were grown at 37° C. in TYP (ampicillin 100 μg/ml) medium to OD₆₀₀ of 0.4 before inducing protein expression with 0.5 mM IPTG. Cells were harvested three hours post-induction by centrifugation for 30 minutes at 400 rpm in a Beckman J-6B. Cell pellets were re-suspended in a buffer containing 50 mM Tris-HCl, 25% (w/v) sucrose, 1 mM NaEDTA, 0.1% (w/v) NaAzide, 10 mM DTT, pH 8.0. After an overnight freeze-thaw step, re-suspended cells were sonicated in 1 minute bursts for a total of around 10 minutes in a Milsonix XL2020 sonicator using a standard 12 mm diameter probe. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl, 0.5% Triton-X100, 200 mM NaCI, 10 mM NaEDTA, 0.1% (w/v) NaAzide, 2 mM DTT, pH 8.0) before being pelleted by centrifugation for 15 minutes at 13000 rpm in a Beckman J2-21. Detergent and salt was then removed by a similar wash in the following buffer: 50 mM Tris-HCl, 1 mM NaEDTA, 0.1% (w/v) NaAzide, 2 mM DTT, pH 8.0. Finally, the inclusion bodies were divided into 30 mg aliquots and frozen at −70° C. Inclusion body protein yield was quantitated by solubilising with 6M guanidine-HCl and measurement with a Bradford dye-binding assay (PerBio).

In order to refold the expressed TCR chains 30 mg of the solubilised TCR β-chain inclusion body and 60 mg of the corresponding solubilised TCR α-chain inclusion body was thawed from frozen stocks. The inclusion bodies were diluted to a final concentration of 5 mg/ml in 6M guanidine solution, and DTT (2M stock) was added to a final concentration of 10 mM. The mixture was incubated at 37° C. for 30 min. Refolding of soluble TCRs: 1 L refolding buffer was stirred vigorously at 5° C.±3° C. The redox couple (2-mercaptoethylamine and cystamine (to final concentrations of 6.6 mM and 3.7 mM, respectively) were added approximately 5 minutes before addition of the denatured TCR chains. The protein was then allowed to refold for approximately 5 hours±15 minutes with stirring at 5° C.±3° C.

Dialysis of Refolded Soluble TCRs:

The refolded TCR was dialysed in Spectrapor 1 membrane (Spectrum; Product No. 132670) against 10 L 10 mM Tris pH 8.1 at 5° C.±3° C. for 18-20 hours. After this time, the dialysis buffer was changed to fresh 10 mM Tris pH 8.1 (10 L) and dialysis was continued at 5° C.±3° C. for another 20-22 hours.

sTCR was separated from degradation products and impurities by loading the dialysed refold onto a POROS 50HQ anion exchange column and eluting bound protein with a gradient of 0-500 mM NaCl over 50 column volumes using an Akta purifier (Pharmacia). Peak fractions were stored at 4° C. and analysed by Coomassie-stained SDS-PAGE before being pooled and concentrated. Finally, the sTCR was purified and characterised using a Superdex 200HR gel filtration column pre-equilibrated in HBS-EP buffer (10 mM HEPES pH 7.4, 150 mM NaCl, 3.5 mM EDTA, 0.05% nonidet p40). The peak eluting at a relative molecular weight of approximately 50 kDa was then pooled and concentrated.

### Example 6

### Diimerisation of TCRs Using a 3.4 kdMal-PEG-Mal Linker

NY-ESO TCRs containing a novel disulphide inter-chain bond, and an additional cysteine residue on the C-terminus of the β-chain (prepared as described above) were cross-linked using non-branched bifunctional maleimide-PEG (MAL-PEG-MAL, MW 3.4 KD, Shearwater corp.). The maleimide groups on the termini of this linker confer free thiol binding specificity to the linker. Prior to cross-linking the TCR was pre-treated with a reducing agent, 0.2 mM DTT (room temperature, overnight), in order to reduce the free cysteine on the soluble TCRs α chains without reducing the disulphide TCR interchain bonds. The soluble TCRs were then re-purified by gel-filtration chromatography (Superdex 75) in PBS buffer containing 10 mM EDTA. Cross-linking was achieved by adding MAL-PEG-MAL (10 mM in DMF) at an approximately 2:1 (protein to cross-linker) molar ratio and subsequently incubating overnight at room temperature. The product was then purified using Superdex 75 HR10/30 gel-filtration column pre-equilibrated in PBS (FIG. 9). Three distinct peaks were observed after the cross-linking, of which one corresponded with the position of intact “monomeric” TCR and the other two corresponded with higher molecular mass species. The material in the peaks was further analysed by SDS-PAGE.

Samples from the three peaks illustrated in FIG. 9 were pre-treated with standard SDS sample buffer (BioRad) without DTT (non-reducing) or with 15 mM DTT (reducing), and were run on a gradient 4-20% PAGE and stained with Coomassie blue stain. Under non-reducing conditions, the material in the three peaks (left to right) appeared as the cross-linked (TCR-PEG-TCR) species (fractions 4&5 corresponding to peak1), an intermediate species (TCR-PEG) (fraction 6/peak2) and the non-modified TCR (fraction7/peak3) respectively. Under the reducing conditions (which cause the disruption of the inter-chain disulphide bond, hence separation of alpha and beta polypeptide chains on SDS-PAGE), the same samples showed cross-linked beta chain dimers (beta-PEG-beta), an intermediate (PEG-beta) and the non-modified beta chain, respectively, while the free alpha chain is distributed (proportionally to protein load) in all fractions (FIG. 10)

The above method was also used to produce A6 TCRs containing a novel disulphide inter-chain bond, and an additional cysteine residue on the C-terminus of the β-chain cross-linked using non-branched bifunctional maleimide-PEG (MAL-PEG-MAL, MW 3.4 KD, Shearwater corp.) (Data not shown)

### Example 7

### BIAcore Surface Plasmon Resonance Characterisation of Divalent A6 TCR and NY-ESO TCR 3.4 KD Mal-PEG-Mal Complexes Binding to Specific pMHC

A surface plasmon resonance biosensor (BIAcore 3000™) was used to analyse the binding of the divalent A6 and NY-ESO TCR PEG complexes to their cognate peptide-MHC ligands. This was facilitated by producing single pMHC complexes (described below) which were immobilised to a streptavidin-coated binding surface in a semi-oriented fashion. Manual injection of pMHC complex allows the precise level of immobilised class I molecules to be manipulated easily.

Such immobilised pMHC complexes are capable of binding both T-cell receptors and the coreceptor CD8αα, both of which may be injected in the soluble phase.

Biotinylated class I HLA-A2—peptide complexes were refolded in vitro from bacterially-expressed inclusion bodies containing the constituent subunit proteins and synthetic peptide, followed by purification and in vitro enzymatic biotinylation (O'Callaghan et al. (1999) Anal. Biochem. 266: 9-15). HLA-heavy chain was expressed with a C-terminal biotinylation tag which replaces the transmembrane and cytoplasmic domains of the protein in an appropriate construct. Inclusion body expression levels of ˜75 mg/litre bacterial culture were obtained. The HLA light-chain or β2-microglobulin was also expressed as inclusion bodies in E. coli from an appropriate construct, at a level of ˜500 mg/litre bacterial culture.

E. coli cells were lysed and inclusion bodies are purified to approximately 80% purity. Protein from inclusion bodies was denatured in 6 M guanidine-HCl, 50 mM Tris pH 8.1, 100 mM NaCl, 10 mM DTT, 10 mM EDTA, and was refolded at a concentration of 30 mg/litre heavy chain, 30 mg/litre β2m into 0.4 M L-Arginine-HCl, 100 mM Tris pH 8.1, 3.7 mM cystamine, mM cysteamine, 4 mg/ml peptide (e.g. tax 11-19), by addition of a single pulse of denatured protein into refold buffer at <5° C. Refolding was allowed to reach completion at 4° C. for at least 1 hour.

Buffer was exchanged by dialysis in 10 volumes of 10 mM Tris pH 8.1. Two changes of buffer were necessary to reduce the ionic strength of the solution sufficiently. The protein solution was then filtered through a 1.5 μm cellulose acetate filter and loaded onto a POROS 50HQ anion exchange column (8 ml bed volume). Protein was eluted with a linear 0-500 mM NaCl gradient. HLA-A2-peptide complex eluted at approximately 250 mM NaCl, and peak fractions were collected, a cocktail of protease inhibitors (Calbiochem) was added and the fractions were chilled on ice.

Biotinylation tagged pMHC complexes were buffer exchanged into 10 mM Tris pH 8.1, 5 mM NaCl using a Pharmacia fast desalting column equilibrated in the same buffer. Immediately upon elution, the protein-containing fractions were chilled on ice and protease inhibitor cocktail (Calbiochem) was added. Biotinylation reagents were then added: 1 mM biotin, 5 mM ATP (buffered to pH 8), 7.5 mM MgCl2, and 5 μg/ml BirA enzyme (purified according to O'Callaghan et al. (1999) Anal. Biochem. 266: 9-15). The mixture was then allowed to incubate at room temperature overnight.

Biotinylated pMHC complexes were purified using gel filtration chromatography. A Pharmacia Superdex 75 HR 10/30 column was pre-equilibrated with filtered PBS and 1 ml of the biotinylation reaction mixture was loaded and the column was developed with PBS at 0.5 ml/min. Biotinylated pMHC complexes eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated pMHC complexes were stored frozen at −20° C. Streptavidin was immobilised by standard amine coupling methods.

The interactions between divalent A6 TCR and NY-ESO TCR 3.4 KD Mal-PEG-Mal complexes, or the respective monomeric soluble TCRs, with their cognate pMHC complex were analysed on a BIAcore 3000™ surface plasmon resonance (SPR) biosensor. SPR measures changes in refractive index expressed in response units (RU) near a sensor surface within a small flow cell, a principle that can be used to detect receptor ligand interactions and to analyse their affinity and kinetic parameters. The probe flow cells were prepared by immobilising the HLA-A2 peptide complexes (1000 response units) in separate flow cells via binding between the biotin cross linked onto β2m and streptavidin which have been chemically cross linked to the activated surface of the flow cells. The assay was then performed by passing sTCR, or the divalent A6 PEG complexes, over the surfaces of the different flow cells at a constant flow rate, measuring the SPR response in doing so. Injections of soluble sTCR at constant flow rate and different concentrations over the peptide-MHC complex were used to define the background resonance.

A streptavidin-coated Biacore chip was loaded with biotinylated HLA A2 refolded in the presence of NY-ESO peptide (1000 response units). A series of dilutions of NY-ESO TCR was then injected and the response measured (FIG. 11a). Dilutions of TCR-PEG-TCR dimers were injected in the same way except longer dissociation phase was allowed (FIG. 11b). The values for affinity (Kd) and dissociation half-time were calculated using Origin software. The dimers exhibited a dramatic avidity effect resulting in a 20× increase in dissociation half-time compared with free NY-ESO TCR.

The intrinsic A6 TCR/HLA binding affinity is relatively high (Kd˜1 uM). A series of dilutions of A6TCR was then injected and the response measured. (FIG. 12a) Dilutions of divalent A6 TCR 3.4 KD Mal-PEG-Mal complexes were injected in the same way except longer dissociation phase was allowed (FIG. 12b). The divalent A6 TCR 3.4 KD Mal-PEG-Mal complexes exhibited a dramatic increase in the stability of the complex due to the avidity effects. (No less than 50% of the material remained bound after 10 minutes of dissociation.) A single injection followed by a prolonged dissociation phase was used to measure the dissociation half-time (FIG. 12c).

The divalent A6 TCR and NY-ESO TCR 3.4 KD Mal-PEG-Mal complexes had disassociation half-lives of 35-79 minutes and 81-90 seconds respectively. These compare to disassociation half-lives of 10.4 and 4.1 seconds respectively for the monomeric soluble A6 and NY-ESO TCRs.

### Example 8

### Design, Expression and Testing of a Single-Chain A6 TCR Incorporating a Novel Disulphide Inter-Chain Bond

The present example details the methods used in the production of a single-chain A6 TCR incorporating a novel disulphide inter-chain bond. Single-chain constructs of this design could also be used as the TCR monomers for the production of divalent TCR-PEG complexes using the methods described in Example 6.

The expression vectors containing the DNA sequences of the mutated A6 TCR α and β chains incorporating the additional cysteine residues required for the formation of a novel disulphide prepared in Example 1 and as shown in FIGS. 1a and 1b were used as the basis for the production of a single-chain A6 TCR, with the exception that the stop codon (TAA) was removed from the end of the α chain sequence, as follows:

The scDiS A6 TCR contains a 30 amino acid linker sequence between the C-terminus of the TCR α chain and the N-terminus of the β chain. FIG. 13 shows the DNA and amino acid sequence of this linker. The cloning strategy employed to produce the scDiS A6 TCR is summarised in FIG. 14.

Briefly, the alpha and beta chains of the A6 dsTCR were amplified by PCR using primers containing restriction sites as shown in FIG. 14, ie.:

Alpha 5′ primer:
ccaaggccatatgcagaaggaagtggagcagaactct
Alpha 3′ primer:
ttgggcccgccggatccgcccccgggggaactttctgggctgggg
Beta 5′ primer:
tcccccgggggcggatccggcgggcccaacgctggtgtcactcag
Beta 3′ primer:
gggaagcttagtctgctctaccccaggcctcg

The two fragments thus generated were PCR stitched using the 5′ alpha and 3′ beta primers to give a single-chain TCR with a short linker containing the sites XmaI-BamHI-ApaI. This fragment was cloned into pGMT7. The full length linker was then inserted in two stages, firstly a 42 bp fragment was inserted using the XmaI and BamHI sites:

5′-CC GGG GGT GGC TCT GGC GGT GGC GGT TCA GGC GGT
GGC G-3′
3′-C CCA CCG AGA CCG CCA CCG CCA AGT CCG CCA CCG
CCT AG-5′

Secondly, a 48 bp fragment was inserted using the BamHI and ApaI sites to create a 90 bp linker between the 3′ end of the alpha chain and the 5′ end of the beta chain. The 48 bp fragment was made by PCR extension of a mixture of the following oligos:

5′- GC GGA TCC GGC GGT GGC GGT TCG GGT GGC GGT GGC
TC-3′
3′- CCA AGC CCA CCG CCA CCG AGT CCG CCA CCG CCC
GGG TG -5′

The product of this extension was digested with BamHI and ApaI and ligated into the digested plasmid containing the 42 bp linker fragment.

The complete DNA and amino acid sequence of the scDiS A6 TCR is shown in FIGS. 15a and 15b respectively.

An additional cysteine codon can be added immediately prior to the ‘stop’ codon at the 3′ terminus of the DNA encoding the above A6 scTCR to produce a molecule suitable for dimer production as described in Example 6.

Expression and Purification of Single-Chain Disulphide Linked A6 TCR:

The expression plasmid containing the single-chain disulphide linked A6 TCR was transformed into E. coli strain BL21pLysS, and single ampicillin-resistant colonies were grown at 37° C. in TYP (ampicillin 100 μg/ml) medium to OD₆₀₀ of 0.4 before inducing protein expression with 0.5 mM IPTG. Cells were harvested three hours post-induction by centrifugation for 30 minutes at 4000 rpm in a Beckman J-6B. Cell pellets were re-suspended in a buffer containing 50 mM Tris-HCl, 25% (w/v) sucrose, 1 mM NaEDTA, 0.1% (w/v) NaAzide, 10 mM DTT, pH 8.0. After an overnight freeze-thaw step, re-suspended cells were sonicated in 1 minute bursts for a total of around 10 minutes in a Milsonix XL2020 sonicator using a standard 12 mm diameter probe. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl, 0.5% Triton-X100, 200 mM NaCl, 10 mM NaEDTA, 0.1% (w/v) NaAzide, 2 mM DTT, pH 8.0) before being pelleted by centrifugation for 15 minutes at 13000 rpm in a Beckman J2-21. Detergent and salt was then removed by a similar wash in the following buffer: 50 mM Tris-HCl, 1 mM NaEDTA, 0.1% (w/v) NaAzide, 2 mM DTT, pH 8.0. Finally, the inclusion bodies were divided into 30 mg aliquots and frozen at −70° C. Inclusion body protein yield was quantitated by solubilising with 6M guanidine-HCl and measurement with a Bradford dye-binding assay (PerBio).

Approximately 15 mg of the solubilised inclusion body chain was thawed from frozen stocks. The inclusion bodies were diluted to a final concentration of 5 mg/ml in 6M guanidine solution, and DTT (2M stock) was added to a final concentration of 100 mM. The mixture was incubated at 37° C. for 30 min. 1 litre of the following refolding buffer: 100 mM Tris pH 8.5, 400 mM L-Arginine, 2 mM EDTA, 5 mM reduced Glutathione, 0.5 mM oxidised Glutathione, 5M urea, 0.2 mM PMSF was prepared and stirred vigoursly at 5° C.+3° C. The redox couple (2-mercaptoethylamine and cystamine (to final concentrations of 6.6 mM and 3.7 mM, respectively) were added approximately 5 minutes before addition of the denatured TCR chains. The protein was then allowed to refold for approximately 5 hours +15 minutes with stirring at 5° C.+3° C. The refold was then dialysed twice, firstly against 10 litres of 100 mM urea, secondly against 10 litres of 10 mM urea, 100 mM Tris pH 8.0. Both refolding and dialysis steps were carried out at 6-8° C.

scTCR was separated from degradation products and impurities by loading the dialysed refold onto a POROS 50HQ anion exchange column and eluting bound protein with a gradient of 0-500 mM NaCl over 50 column volumes using an Akta purifier (Pharmacia). Peak fractions were stored at 4° C. and analysed by Coomassie-stained SDS-PAGE before being pooled and concentrated. The sTCR was then purified and characterised using a Superdex 200HR gel filtration column (FIG. 8) pre-equilibrated in HBS-EP buffer (10 mM HEPES pH 7.4, 150 mM NaCl, 3.5 mM EDTA, 0.05% nonidet p40). Peak fractions were stored at 4° C. and analysed by Coomassie-stained SDS-PAGE before being pooled and concentrated.

### Example 9

### Iodine Radio-Labelling of NY-ESO TCR Dimers

PEGylated divalent NY-ESO TCR 3.4 kd Mal-PEG-Mal complexes, prepared as described in Example 6 were labelled with ¹²⁵I as follows.

50 μl 1.0 M phosphate buffer was added to 100 μl 1 mg/ml divalent NY-ESO TCR 3.4 KD Mal-PEG-Mal complexes in PBS. 15 μl (1500 μCi) of ¹²⁵I iodide solution (Amersham Bioscience, UK) was then added to this protein solution followed by 50 μl 2 mg/ml chloramine-T (Sigma). The solution was then mixed and left at room temperature for 1 minute. 50 μl 1 mg/ml L-tyrosine solution was then added and the solution was then mixed thoroughly. 2.3 ml 0.05 M phosphate buffer was then added and the solution was then added to a P10 column (Sigma). The void volume of the column was then collected. Twelve 0.5 ml aliquots of 0.05 M phosphate buffer were then added to the column and the eluted fractions were collected and numbered. A further 5 ml 0.05M phosphate buffer was then added to the column and the resultant eluted fraction was collected.

The activity in the collected fractions was then estimated by scintillation counting. TLC was then carried out on samples from each of the fractions to allow quantitation of the protein/iodine content present.

P10 column fractions 1-6 were combined to give a volume of approximately 3.5 ml. 1.84 ml 54 μg/ml divalent NY-ESO TCR 3.4 KD Mal-PEG-Mal complexes in PBS was then added to give 10 mg total protein. A further 4.7 ml of PBS was added to the solution, which was then counted in a scintillation counter. The solution was then diluted to 10 ml with PBS.

The ¹²⁵I used to label the TCR in the present example for imaging purposes could be replaced with ¹³¹I in order to produce a therapeutic agent.

### Example 10

### In-Vivo Tumour Targeted Using Divalent NY-ESO TCR 3.4 KD Mal-PEG-Mal Complexes

The ability of ¹²⁵I-labelled PEGylated divalent NY-ESO TCR 3.4 KD Mal-PEG-Mal complexes, prepared as described in Example 9 to localise to a tumour expressing HLA-A2 NY-ESO was investigated as follows:

12 female Nude rats (HARLAN, France) were used in this trial

6 of the rats were s.c. injected with a melanoma tumour-forming cell line (A375-SM) and left for 15-20 days to allow subcutaneous tumour development (Test Group).

The remainder of the rats were not injected with the tumour-forming cell-line (Control Group).

The rats then received the following i.v. bolus dosage of the divalent NY-ESO TCR 3.4 KD Mal-PEG-Mal complexes:

2 mg/kg divalent NY-ESO TCR 3.4 KD Mal-PEG-Mal complexes in PBS (˜25 μCi/rat)

1 Test Group and 1 Control Group rat were then sacrificed at the following time points after divalent NY-ESO TCR 3.4 kd Mal-PEG-Mal complexes administration:

20 minutes

1 hour

3 hours

6 hours

24 hours

48 hours

These rats were then subjected to Quantitative Whole Body Autoradiography (QWBA) using standard procedures. Sections were presented at five levels of the rat bodies to include as many tissues as feasible. The quantitative distribution of radioactivity was then carried out using a Fuji BAS 1500 bio-image analyser and the associated Tina and SeeScan software.

Results

The distribution of the radio-labelled divalent NY-ESO TCR 3.4 KD Mal-PEG-Mal complexes at each time-point was visualised by autoradiography. The radio-labelled dimer initially spread quickly throughout the rats bodies. This can be seen in FIG. 16 which is an autoradiograph of a rat sacrificed 20 minutes after dimer administration. The longer-term ability of the dimer to cause localisation to, and within, the tumour can clearly be seen in FIG. 17, which is an autoradiograph of a rat sacrificed 48 hours after dimer administration

Conclusions

The distribution of radioactivity at 48 hours afterdivalent NY-ESO TCR 3.4 KD Mal-PEG-Mal complexes administration indicates that the radio-labelled dimer has localised to, and penetrated the tumour.

### Example 11

### Tetramerisation of TCRs

A6 TCRs containing a novel disulphide inter-chain bond, and an additional cysteine residue on the C-terminus of the β-chain (prepared as described above) were tetramerised using a tetrameric maleimide-PEG (4 arm MAL-PEG, MW 20 KD, Shearwater Corporation). The maleimide groups on the termini of this linker confer free thiol binding specificity to the linker. Prior to tetramerisation the TCR was pre-treated with a reducing agent, 0.5 mM DTT (37° C., 1 hour), in order to reduce the free cysteine on the soluble TCRs β chains without reducing the disulphide TCR inter-chain bonds. The soluble TCRs were then re-purified by gel-filtration chromatography (Superdex 75) in PBS buffer containing 10 mM EDTA. Tetramerisaton was achieved by adding the 4 arm MAL-PEG (10 mM in DMF) at an approximately 4:1 (protein to cross-linker) molar ratio and subsequent incubation overnight at room temperature. The product was then purified using Superdex 75 HR10/30 gel-filtration column pre-equilibrated in PBS. The eluted fractions were further analysed by SDS-PAGE. Samples from the fractions were pre-treated with standard SDS sample buffer (BioRad) without DTT (non-reducing) or with 15 mM DTT (reducing), and were run on a gradient 4-20% PAGE and stained with Coomassie blue stain.

### Example 12

### BIAcore Surface Plasmon Resonance Characterisation of Tetravalent A6 TCR PEG Complexes Binding to Specific pMHC

The interactions between a tetravalent A6 TCR PEG complex, prepared as described in Example 11, with it's cognate pMHC complex was analysed on a BIAcore 3000™ surface plasmon resonance (SPR) biosensor using the methods described in Example 7.

Briefly, a streptavidin-coated BIAcore chip was loaded with biotinylated HLA A2 refolded in the presence of Tax peptide (1000 response units). The tetramers exhibited a dramatic avidity effect, to the extent that calculation of a disassociation half-life proved problematic. A comparison of FIGS. 12c and 18 illustrate the these A6 TCR tetramers have increased avidity, and therefore longer disassociation half-lives than the corresponding A6 TCR dimers.

### Example 13

### Cryostat and Paraffin Preparation of Tumour Sections

In order to carry out Immuno-histochemical (IHC) and Immunofluorescent (IF) studies tumours were removed from three of the rats used in Example 10. The tumours were cut in half and then one half was prepared by formalin-fixed paraffin embedding and the other half by cryostat preparation using the following methods:

Cryostat Preparation

The tumour samples were snap frozen in liquid nitrogen and then sliced into 6 μm sections using a cryostat. These sections were used for IF studies.

Formalin-Fixed Paraffin-Embedded Tissue Preparation

The remaining tumour samples were fixed in 10% neutral formalin and embedded in paraffin wax. 3 μm sections were then sliced from the embedded tumour sections using a microtome. These sections were used for IHC studies.

### Example 14

### IHC and IF Staining and Visualisation

TCR β Chain and NY-ESO TCR-Specific Staining

The distribution of both all TCR β chains and NY-ESO TCR was visualised in formalin-fixed paraffin-embedded tumour tissue samples. These were carried out using an indirect IHC method with a horse-radish peroxidase enzyme marker as follows:

Protocol

1. Sections were warmed for 10 minutes at 40 C.

2. Sections were deparaffinised in Histoclear for 10 minutes and re-hydrate by immersing for 5 minutes each in 100% Industrial Methylated Spirit (IMS), 70% IMS/H₂O, H₂O.

3. The sections were washed in PBS for 5 minutes.

4. Endogenous peroxidase activity was removed from the sections by immersion in 0.3% H₂O₂ for 30 minutes at room temperature.

5. The sections were washed three times in PBS for 5 minutes each time.

6. 100 μl of blocking serum was added to each section and left for 30 minutes. The blocking serum is prepared from the species in which the secondary antibody is raised. This step is carried out to block non-specific binding of mouse IgG to the section so that when the secondary Ab is applied, it only binds to the primary antibody.

7. 100 μl of the primary Ab (rabbit polyclonal anti-TCR antibody) was added to each section and covered with a cover slip. A 1:50 dilution in the blocking serum that was used in step 6 was made and this was left for 40-50 minutes at room temperature.

8. The sections were washed twice in PBS for 5 minutes each time.

9. 100 μl of the biotinylated secondary Ab (anti-rabbit antibody) was added to each section, the sections were then covered with a cover slip and left for 30 minutes.

10. The sections were washed twice in PBS for 5 minutes each time.

11. Add 100 μl of Avadin/Biotin Complex (made up according to the manufacturers instructions 30 minutes before use (Vectastain kit) to each section for 30 minutes at room temperature.

12. The sections were washed twice in PBS for 5 minutes each time.

13. 100 μl of enzyme substrate, diaminobenzidine tetrahydrochloride (DAB), was added to each section and left for 5 minutes.

14. The sections were then rinsed in tap water

15. The sections were counterstained with haematoxylin for 15 seconds

16. The sections were de-hydrated by immersion for 5 minutes in 70% IMS/dH₂O, 100% IMS and then 5-10 minutes in Histoclear.

17. Mount with DPX.

Two different controls were included in these TCR-staining studies. Firstly, “omission controls” in which the primary antibody was omitted. Secondly, anti-NY-ESO TCR antibody pre-absorbed to soluble NY-ESO TCR was also used.

HLA-A2 Staining

The distribution of HLA-A2 within the cryostat prepared tumour sections was evaluated by a standard Immunofluorescent technique. Briefly, the cryostat-prepared sections to be imaged were bathed in a saturating concentration of an anti-HLA-A2 antibody that was labelled with a FITC fluorescent marker. The excess unbound antibody was then washed off and the sample was then prepared for imaging. This method was also repeated using a FITC-labelled non-specific IgG as a control.

Haemotoxylin and Eosin (H&E) Staining

H&E staining was carried out on formalin-fixed, paraffin-embedded tumour sections using the following method:

1. Sections were deparaffinised in Histoclear for 10 minutes and re-hydrate by immersing for 5 minutes each in 100% (IMS), 70% IMS/H₂O, H₂O.

2. Sections were immersed in haematoxylin for 10-15 minutes.

3. Sections were thoroughly washed in tap water and then in distilled H₂O.

4. The slides were de-stained by dipping for a few seconds in acid/alcohol (1% HCl/70% IMS).

5. The slides were de-hydrated by dipping for 2 minutes in each of 70% IMS/H₂O and 100% IMS.

6. The slides were dipped in eosin for 15-30 seconds.

7. The slides were immersed in 100% IMS for 2 minutes.

8. The slides were immersed in 100% IMS for 5 minutes

9. The slides were immersed in Histoclear for 10 minutes

10. Finally the slides were mounted with DPX

The stained tumour sections were then imaged using light (H&E stain) or fluorescence (TCR/HLA stains) microscopy.

Results

The results of the NY-ESO TCR staining carried out on tumour sections prepared from rats sacrificed 20 minutes after injection of the NY-ESO PEG dimer demonstrate that the TCR dimer has rapidly penetrated the tumour mass. (see FIGS. 20a-20d)

NY-ESO TCR distribution was directly compared in the viable and necrotic tissue (determined by H&E staining) within the tumour. This comparison revealed that NY-ESO TCR was predominately found within the viable areas of the tumour. (See FIG. 21)

HLA-A2 distribution was directly compared in the viable and necrotic tissue (determined by H&E staining) within the tumour. This comparison revealed that HLA-A2 was predominately found within the viable areas of the tumour. (See FIGS. 19a-19c)

Conclusions

The above experiment shows that the TCR PEG dimers have penetrated into the tumours within 20 minutes post-injection. FIGS. 20a-20d indicate that the TCR PEG dimers may have been internalised into the tumour cells. The very short timescale over which this appears to have occurred indicates an active ingress mechanism. Furthermore, the injected NY-ESO TCR PEG was predominately found in the viable regions of the tumour. Since the HLA-A2 distribution is largely confined to the viable areas of the tumours, this area of tumours is expected to be selectively targeted.

### Example 15

### Production of High Affinity Soluble Heterodimeric A6 TCR with Non-Native Disulfide Bond Between Constant Regions, Containing CDR3 Mutations

A high affinity soluble heterodimeric A6 TCR was expressed and refolded using the methods described in Example 5, This soluble A6 TCR contains the TCR α chain previously described, except that a cysteine residue was added to the C terminal of this chain to facilitate dimerisation. (See FIGS. 22a and 22a for the DNA and amino acid sequences respectively) The β chain of this TCR contains mutations in the Complimentarity Determining Region 3 (CDR 3), which confer increased affinity for its cognate HLA-A2-Tax ligand. (see FIGS. 23a and 23b for the DNA and amino acid sequences respectively)

### Example 16

### Production and BIAcore Testing of a Divalent High Affinity A6 TCR PEG Complex

A PEG dimer of the high affinity soluble heterodimeric A6 TCR produced as described in Example 15 was prepared using the methods as described in Example 6.

The binding of the high affinity A6 TCR and the high affinity A6 TCR PEG dimer to their cognate ligand was then assessed using a BIAcore 3000™. The methods used were similar to those used in Example 7 except that the high affinity of this mutant A6 TCR necessitated that binding studies were carried out using a single injection. The conditions used, optimised for kinetic studies, are summarised below:

BIAcore Conditions

Buffer: PBS; Flow rate: 50 ml/min

Ligand (HLA-A2 Tax) immobilization level: 500 RU

Injection (Kinject): 250 ml association/2400 ml dissociation

Protein Samples

High affinity (HA) A6 TCR; 4.4 A280/ml in PBS (15.05.03NL); working dilution: 4 ml in 400 ml final;

High affinity A6 TCR dimer (TCR-PEG-TCR; 3,400 K linear PEG); 1.0 A280/ml in PBS; working dilution: 17.6 ml in 400 ml final

Results

The T_{1/2} for the interaction between the high affinity A6 TCR and HLA-A2 Tax was calculated to be 41 minutes. (See FIG. 24) The T_{1/2} for the interaction between the high affinity A6 TCR 3.4 KD PEG dimer and HLA-A2 Tax was calculated to be in the order of 22 to 78 hours. (See FIG. 25)

Conclusions

As stated above the dimerisation of the high affinity A6 TCR increased the interaction T_{1/2} from 41 minutes to between 22-78 hours. The use of the high affinity mutant A6 TCR in a PEG dimer increased the interaction T_{1/2} from 35-79 minutes (native A6 TCR PEG dimer) to 22-78 hours.

### Example 17

### Dimerisation of High Affinity A6 TCRs Using a 20 KD Mal-PEG-Mal Linker

High affinity A6 TCRs containing a novel disulphide inter-chain bond, and an additional cysteine residue on the C-terminus of the β-chain (prepared as described above) were cross-linked using non-branched bifunctional maleimide-PEG (MAL-PEG-MAL, MW 20 KD, NOF Corp.) using the production method described in Example 6.

The dimeric high affinity A6 TCR 20 KD Mal-PEG-Mal complexes produced were then re-purified using an altered method as follows:

Anion-exchange chromatography was used to carry out the initial re-purification step for the high affinity A6 TCR 20 KD Mal-PEG-Mal complexes:

Column: MonoQ HR5/5 high resolution column (Pharmacia))

Buffer: 25 mM Tris-HCL, pH8, 0.5 mM EDTA.

Elution gradient: 0-0.5M NaCl (in the above buffer).

The dimer eluted in the middle of the gradient (at approxiamtatly 0.25M NaCl) as a single peak.

The product then underwent final purification using Superdex 200 HR10/30 gel-filtration column pre-equilibrated in PBS

### Example 18

### BIAcore Surface Plasmon Resonance Characterization of the High Affinity A6 TCR 20 KD Mal-PEG-Mal Complexes Binding to HLA-A2 Tax

The interactions between the high affinity A6 TCR 20 KD Mal-PEG-Mal complexes, produced as described in Example 17, with their cognate pMHC complex (HLA-A2 Tax) was analysed on a BIAcore 3000™ surface plasmon resonance (SPR) biosensor as described in Example 16.

The divalent high affinity A6 TCR 20 KD PEG complexes had a disassociation half-live of 9.5 days with respect to HLA-A2-Tax. This compares to a disassociation half-live of 22-78 hours for the high affinity divalent A6 3.4 KD Mal-PEG-Mal complexes for the same interaction.

Without wishing to be bound by theory, it is believed that the increased disassociation half-life demonstrated for the high affinity A6 TCR 20 KD PEG complexes compared to that of the high affinity A6 TCR 3.4 KD PEG complexes is due to the longer linker providing more opportunity for bivalent attachment to HLA complexes on the surface of the target cells.

### Example 19

### Dimerisation of A6 TCRs Using a 5 KD Forked Mal-PEG-Mal Linker

A6 TCRs containing a novel disulphide inter-chain bond, and an additional cysteine residue on the C-terminus of the β-chain (prepared as described in Example 5) were cross-linked using a 5 KD forked bifunctional maleimide-PEG (L-PEG-MAL, MW 5 KD, Shearwater Corporation) using the production and re-purification methods described in Example 6.

### Example 20

### BIAcore Surface Plasmon Resonance Comparison of the Dimeric A6 TCR 5 KD Forked Mal-PEG-Mal Complex, Dimeric A 6 TCR 3.4 KD Linear Mal-PEG-Mal Complex and a Monomeric 3.4 KD Linear A6 TCR Complex Binding to HLA-A2 Tax

The interactions between the dimeric A6 TCR 5 KD forked Mal-PEG-Mal complexes, produced as described in Example 19, with their cognate pMHC complex (HLA-A2 Tax) was analysed on a BIAcore 3000™ surface plasmon resonance (SPR) biosensor as described in Example 16.

The interaction between the dimeric A6 TCR 3.4 KD linear Mal-PEG-Mal complexes, produced as described in Example 5, and a monomeric A6 TCR 3.4 KD linear PEG complex was also analysed on the same BIAcore chip to provide a comparison.

The divalent A6 TCR 5 KD forked PEG complexes and the divalent A6 3.4 KD Mal-PEG-Mal complexes had similar binding characteristics with respect to HLA-A2-Tax. (See FIG. 26)

### Example 20

### Cell Staining Using High Affinity Clone 134 A6 TCR 20 KD PEG Dimers

PP antigen presenting cells were pulsed with Tax peptide at a range of concentrations (10⁻⁵-10⁻⁹M) for 90 minutes at 37° C. Controls, also using T2 cells were pulsed with 10⁻⁵M Flu peptide or incubated without peptide (unpulsed). After pulsing the cells were washed in serum-free RPMI and 1×10⁵ cells were incubated with high affinity clone 134 A6 TCR 20 KD PEG dimer labelled with Alexa 488 (Molecular probes, The Netherlands) for 10 minutes at room temperature. After washing the cells, the binding of the labelled TCR dimers was examined by flow cytometry using a FACSVantage SE (Becton Dickinson).

Results

As illustrated in FIGS. 27a and 27b specific staining of PP cells by high affinity clone 134 A6 TCR 20 KD PEG dimers could be observed at Tax peptide concentrations of down to 10⁻⁵ M.

1. A multivalent T cell receptor (TCR) complex comprising at least two TCRs, linked by a non-peptidic polymer chain or a peptidic linker sequence.

2. A TCR complex as claimed in claim 1 wherein the TCRs are constituted by amino acid sequences corresponding to extracellular constant and variable region sequences of native TCRs.

3. A TCR complex as claimed in claim 1 wherein the polymer chain or peptidic linker sequence extends between amino acid residues of each TCR which are not located in a variable region sequence of the TCR.

4. A TCR complex as claimed in claim 1 in which the TCRs are linked by a polyalkylene glycol chain or a peptidic linker derived from a human multimerisation domain.

5. A TCR complex as claimed in claim 4 wherein a divalent alkylene spacer radical is located between the polyalkylene glycol chain and its point of attachment to a TCR of the complex.

6. A TCR complex as claimed in claim 5 wherein the divalent alkylene spacer is —CH2- or —CH2CH2-.

7. A TCR complex as claimed in claim 5 wherein the polyalkylene glycol chain is a polyethylene glycol (PEG) chain.

8. A TCR complex as claimed in claim 1 which is divalent.

9. A TCR complex as claimed in claim 1 which is trivalent.

10. A TCR complex as claimed in claim 1 which is tetravalent.

11. A TCR complex as claimed in claim 7 in which two TCRs are linked by a linear PEG chain.

12. A TCR complex as claimed in claim 7 in which more than two TCRs are linked by a branched linear PEG chain.

13. A TCR complex as claimed in claim 7 in which three or four TCRs are linked by a branched PEG chain.

14. A TCR complex as claimed in claim 1 wherein at least one TCR is a single chain T-cell receptor (scTCR) polypeptide.

15. A TCR complex as claimed in claim 14, wherein the scTCR is constituted by TCR amino acid sequences corresponding to extracellular constant and variable region sequences present in native TCR chains and a linker sequence, the latter linking a variable region sequence corresponding to that of one chain of a native TCR to a constant region sequence corresponding to a constant region sequence of another native TCR chain;
the variable region sequences of the scTCR polypeptide are mutually orientated substantially as in native TCRs; and
a disulfide bond which has no equivalent in native T cell receptors links residues of the polypeptide.

16. A TCR complex as claimed in claim 15 wherein the scTCR polypeptide has
a first segment constituted by an amino acid sequence corresponding to a TCR α or β chain variable region sequence fused to the N terminus of an amino acid sequence corresponding to a TCR α chain constant region extracellular sequence,
a second segment constituted by an amino acid sequence corresponding to a TCR β or γ chain variable region fused to the N terminus of an amino acid sequence corresponding to a TCR β chain constant region extracellular sequence,
a linker sequence linking the C terminus of the first segment to the N terminus of the second segment, or vice versa, and
a disulfide bond between the first and second chains, said disulfide bond being one which has no equivalent in native αβ or γδ T cell receptors,
the length of the linker sequence and the position of the disulfide bond being such that the variable region sequences of the first and second segments are mutually orientated substantially as in native αγ or γδ T cell receptors.

17. A TCR complex as claimed in claim 16 wherein the linker sequence has the formula -P-AA-P- wherein P is proline and AA represents an amino acid sequence wherein the amino acids are glycine and serine.

18. A TCR complex as claimed in claim 16 wherein the linker sequence links the C terminus of the first segment to the N terminus of the second segment.

19. A TCR complex as claimed in claim 18 wherein the linker sequence consists of from 26 to 41 amino acids.

20. A TCR complex as claimed in claim 18 wherein the linker sequence consists of 29, 30, 31 or 32 amino acids.

21. A TCR complex as claimed in claim 18 wherein the linker sequence consists of 33, 34, 35 or 36 amino acids.

22. A TCR complex as claimed in claim 18 wherein the linker sequence has the formula -PGGG-(SGGGG)₅-P- wherein P is proline, G is glycine and S is serine.

23. A TCR complex as claimed in claim 18 wherein the linker sequence has the formula -PGGG-(SGGGG)₆-P- wherein P is proline, G is glycine and S is serine.

24. A TCR complex as claimed in claim 1 wherein at least one TCR is a dimeric T-cell receptor (dTCR) polypeptide pair.

25. A TCR complex as claimed in claim 1 wherein each TCR is a dimeric T-cell receptor (dTCR) polypeptide pair.

26. A TCR complex as claimed in claim 24, wherein the or each dTCR polypeptide pair is constituted by TCR amino acid sequences corresponding to extracellular constant and variable region sequences present in native TCR chains, and the variable region sequences are mutually orientated substantially as in native TCRs.

27. A TCR complex as claimed in claim 26 wherein the or each dTCR polypeptide pair is constituted by
a first polypeptide wherein a sequence corresponding to a TCR α or β chain variable region sequence fused to the N terminus of a sequence corresponding to a TCR α chain constant region extracellular sequence, and
a second polypeptide wherein a sequence corresponding to a TCR β or γ chain variable region sequence fused to the N terminus a sequence corresponding to a TCR β chain constant region extracellular sequence,
the first and second polypeptides being linked by a disulfide bond which has no equivalent in native αβ or γδ T cell receptors.

28. A TCR complex as claimed in claim 13 wherein the or each scTCR polypeptide ordTCR polypeptide pair has amino acid sequences corresponding to αβ TCR extracellular constant and variable region sequences.

29. A TCR complex as claimed in claim 13 wherein the or each scTCR polypeptide or dTCR polypeptide pair has amino acid sequences corresponding to extracellular αβ TCR constant region sequences and γδ TCR variable region sequences.

30. A TCR complex as claimed in claim 13 wherein the or each scTCR polypeptide or dTCR polypeptide pair has amino acid sequences corresponding to non-human extracellular αβ TCR constant region sequences and human TCR variable region sequences.

31. A TCR complex as claimed in claim 13 wherein an amino acid sequence of one member of the or each dTCR polypeptide pair, or an amino acid sequence of the or each scTCR polypeptide, corresponds to a native TCR extracellular constant chain Ig domain sequence.

32. A TCR complex as claimed in claim 13 wherein the or each dTCR polypeptide pair or the or each scTCR polypeptide includes sequences corresponding to native TCR extracellular constant chain Ig domain sequences.

33. A TCR complex as claimed in claim 32 wherein a disulfide bond links amino acid residues of the said constant chain Ig domain sequences, which disulfide bond has no equivalent in native TCRs.

34. A TCR complex as claimed in claim 33 wherein the said disulfide bond is between cysteine residues corresponding to amino acid residues whose β carbon atoms are less than 0.6 nm apart in native TCRs.

35. A TCR complex as claimed in claim 34 wherein the said disulfide bond is between cysteine residues substituted for Thr48 of exon 1 of TRAC*01 and Ser 57 of exon 1 of TRBC1*01 or TRBC2*01 or the non-human equivalent thereof.

36. A TCR complex as claimed in claim 34 wherein the said disulfide bond is between cysteine residues substituted for Thr 45 of exon 1 of TRAC*01 and Ser 77 of exon 1 of TRBC1*01 or TRBC2*01 or the non-human equivalent thereof.

37. A TCR complex as claimed in claim 34 wherein the said disulfide bond is between cysteine residues substituted for Tyr 10 of exon 1 of TRAC*01 and Ser 17 of exon 1 of TRBC1*01 or TRBC2*01 or the non-human equivalent thereof.

38. A TCR complex as claimed in claim 34 wherein the said disulfide bond is between cysteine residues substituted for Thr 45 of exon 1 of TRAC*01 and Asp 59 of exon 1 of TRBC1*01 or TRBC2*01 or the non-human equivalent thereof.

39. A TCR complex as claimed in claim 34 wherein the said disulfide bond is between cysteine residues substituted for Ser 15 of exon 1 of TRAC*01 and Glu 15 of exon 1 of TRBC1*01 or TRBC2*01 or the non-human equivalent thereof.

40. A TCR complex as claimed in claim 32 wherein the sequences corresponding to native TCR extracellular constant chain Ig domain sequences are truncated at their C-termini relative to said native sequences such that the cysteine residues which form the native interchain disulphide bond are excluded.

41. A TCR complex as claimed in claim 32 wherein in the sequences corresponding to native TCR extracellular constant chain Ig domain sequences the cysteine residues which form the native interchain disulphide bond are substituted by non-cysteine residues.

42. A TCR complex as claimed in claim 41 wherein the cysteine residues which form the native interchain disulfide bond are substituted by serine or alanine.

43. A TCR complex as claimed in claim 32 wherein in the or each dTCR or scTCR there is no unpaired cysteine residue corresponding an unpaired cysteine residue present in a native TCR.

44. A TCR complex as claimed in claim 1 which comprises at least two dTCR polypeptide pairs linked by a polyalkylene glycol chain, wherein a divalent alkylene spacer radical is optionally located between the polyalkylene glycol chain and its point of attachment to a dTCR of the complex, and wherein each said dTCR pair is constituted by:
a first polypeptide wherein a sequence corresponding to a TCR α chain variable domain sequence is fused to the N terminus of a sequence corresponding to a TCR α chain constant domain extracellular sequence, and
a second polypeptide wherein a sequence corresponding to a TCR β chain variable domain sequence is fused to the N terminus a sequence corresponding to a TCR β chain constant domain extracellular sequence,
the first and second polypeptides being linked by a disulfide bond between cysteine residues substituted for Thr 48 of exon1 of TRAC*01 and Ser 57 of exon 1 of TRBC1*01 or TRBC2*01 or the non-human equivalent thereof, the point of attachment of the polyalkylene linker to each dTCR of the complex being via the thiol group of a cysteine residue at the C-terminus of the dTCR.

45. A TCR complex as claimed in claim 44 wherein two dTCRs are linked by a polyethylene glycol chain.

46. A TCR complex as claimed in claim 1 which binds a peptide MHC complex.

47. A TCR complex as claimed in claim 1 which binds a given MHC type or types

48. A TCR complex as claimed in claim 1 which binds aCD1-antigen complex.

49. A TCR complex as claimed in claim 1 which binds a superantigen or a peptide-MHC/superantigen complex.

50. A TCR complex as claimed in claim 1 wherein the TCRs are specific for a given TCR ligand, and (i) are mutated in the variable domain(s) relative to the native TCR specific for said TCR ligand, and (ii) have a Kd for the said TCR ligand less than that of the native TCR

51. A TCR complex as claimed in claim 1 wherein the TCRs are specific for a given TCR ligand, and (i) are mutated in the variable domain(s) relative to the native TCR specific for said TCR ligand and (ii) have a Kd for the said TCR ligand less than that of the native TCR as measured by Surface Plasmon Resonance.

52. A TCR complex as claimed in claim 1 wherein the TCRs are specific for a given TCR ligand, and (i) are mutated in the variable domain(s) relative to the native TCR specific for said TCR ligand and (ii) have an off-rate (k_{off}) for the said TCR ligand less than that of the native TCR.

53. A TCR complex as claimed in claim 1 wherein the TCRs are specific for a given TCR ligand, and (i) are mutated in the variable domain(s) relative to the native TCR specific for said TCR ligand and (ii) have an off-rate (k_{off}) for the said TCR ligand less than that of the native TCR as measured by Surface Plasmon Resonance.

54. A TCR complex as claimed in claim 1 associated with a cytotoxic moiety, a detectable or imageable moiety, or an immunostimulatory peptide or polypeptide.

55. A composition comprising a TCR complex as claimed in claim 1, together with a pharmaceutically acceptable carrier.

56. (canceled)

57. (canceled)

58. (canceled)

59. (canceled)

60. A method of inducing apoptosis in a target cell population in a patient in need thereof comprising administering to a patient an amount of a TCR complex as claimed in claim 1 effective to induce said apoptosis.

61. A method of inducing an immune response to a target cell population in a patient in need thereof comprising administering to a patient an amount of a TCR complex as claimed in claim 54 which is associated with an immunostimulatory peptide or polypeptide effective to induce said apoptosis.

62. A method for the treatment of autoimmune disease comprising the administration to a patient of an effective amount of a TCR complex as claimed in claim 1.

63. A method for the treatment of cancer comprising the administration to a patient of an effective amount of a TCR complex as claimed in claim 1.

64. A method of detecting or imaging cells displaying a given TCR ligand comprising contacting said cells with a TCR complex as claimed in claim 1 wherein the TCR present in the complex binds to the said TCR ligand, and said complex has a detectable or imageable moiety associated therewith.

65. A method as claimed in claim 64 wherein said cells are cancerous cells including cancerous cells forming a tumour mass.

66. A method as claimed in claim 64 for the diagnosis or monitoring of a disease characterised by infected cells, or cancer.

67. A method as claimed in claim 64 carried out in-vivo.

68. A method as claimed in claim 64 carried out ex-vivo.

69. A diagnostic or imaging composition for cancerous or infected cells comprising multivalent TCR complex as claimed in claim 1 which has a detectable or imageable moiety associated therewith.
